(19) United States
(12) Patent Application Publication
(10) Pub. No.: US 2016/0168185 A1
(43) Pub. Date: Jun. 16, 2016

(54) NON-RIBOSE CONTAINING INHIBITORS OF HISTONE METHYLTRANSFERASE DOT1L FOR CANCER TREATMENT

(71) Applicant: BAYLOR COLLEGE OF MEDICINE, Houston, TX (US)

(72) Inventors: Yongcheng SONG, Houston, TX (US); Lisheng DENG, Houston, TX (US); Yang YAO, Houston, TX (US); Li ZHANG, Houston, TX (US); Cong WANG, Houston, TX (US); Michele S. REDELL, Houston, TX (US); Shuo DONG, Houston, TX (US)

(73) Assignee: Baylor College of Medicine, Houston, TX (US)

(21) Appl. No.: 14/907,149

(22) PCT Filed: Jul. 22, 2014
(86) PCT No.: PCT/US2014/047577

§ 371 Date: Jan. 22, 2016

Related U.S. Application Data
(60) Provisional application No. 61/857,074, filed on Jul. 22, 2013.
(60) Provisional application No. 61/872,535, filed on Aug. 30, 2013.
(60) Provisional application No. 61/886,885, filed on Oct. 4, 2013.
(60) Provisional application No. 61/886,873, filed on Oct. 4, 2013.

(51) Int. Cl. C07H 19/16; C07D 473/34

(52) U.S. Cl. CPC C07H 19/16; C07D 473/34

(57) ABSTRACT

A compound of Formula I, a pharmaceutically acceptable salt thereof, a prodrug thereof, or combinations thereof:

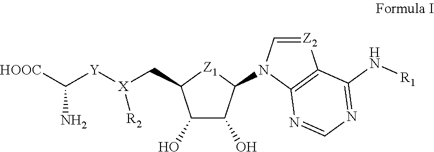

wherein R₁ is H, methyl, or benzyl;
   R₂ is 2-cyanoethyl, 2-methoxycarbonylethyl, or 2-iodoethyl;
   X is N or S; wherein if X=S, R₂=O;
   Y is C₃ or C₄;
   Z₁ is O, S, N, or CH₂; and
   Z₂ is N or CR₄, wherein R₄ is a halogen, alkyl, aryl or a 5- or 6-membered heterocycle; and
wherein said compound is selective for DOT1L Methyl Transferase.

### CROSS REFERENCE TO RELATED APPLICATIONS

This application is the U.S. National Stage entry under 35 U.S.C. §371 of International Patent Application No. PCT/US2014/047577, filed Jul. 22, 2014, and entitled “Non-Ribose Containing Inhibitors of Histone Methyltransferase Dot1L For Cancer Treatment,” which claims priority to U.S. Provisional Application Nos. 61/857,074; 61/872,535; 61/886,885; and 61/886,873 respectively filed Jul. 22, 2013, Aug. 30, 2013, Oct. 4, 2013 and Oct. 4, 2013, all of which are hereby incorporated by reference in their entireties for all purposes.

### STATEMENT REGARDING FEDERALLY SPONSORED RESEARCH OR DEVELOPMENT

Not applicable.

### TECHNICAL FIELD

This disclosure generally relates to compositions and methods for cancer treatment. More specifically, it relates to compositions and methods of using non-ribose containing selective inhibitors of histone methyltransferase DOT1L for the treatment of specific cancers, such as for example leukemia and breast cancer.

### BACKGROUND

The human genome is tightly packed into millions of nucleosomes, which is composed of a short segment of DNA (˜146 base pairs) winding around a disc-like histone core having two copies each of histone H2A, H2B, H3 and H4. Histones are rich of basic amino acid residues lysine and arginine, which not only provide electrostatic interactions with DNA for tight binding, but can be covalently modified. Histone methylation at its lysine sidechains is one of the most studied post-translational modifications. Histone lysine methyltransferases (HKMT) include a large family (>50) of enzymes, many of which have been found to play important roles in cell differentiation, gene regulation, DNA recombination and damage repair. Therefore, small molecule inhibitors of histone methyltransferases (HMTs) are useful chemical probes for these biological studies as well as potential therapeutics. However, development of HMT inhibitors has been in its infancy: very few inhibitors have been discovered and developed.

Histone H3-lysine79 (H3K79) methyltransferase DOT1L is of particular interest, and is a target for inhibitor discovery and development. The full-length human DOT1L contains 1537 amino acids, with its N-terminal ˜360 amino acids being highly conserved from yeast to mammals and identified to be an H3K79 methyltransferase. The remaining C-terminal part of mammalian DOT1L is involved in physical interactions with many transcription relevant proteins such as AF4, 9, 10, ENL. Therefore, the general biological function of DOT1L is to methylate H3K79 as a member of a large protein complex, which can initiate and/or maintain an active transcription state.

DOT1L is an unique HKMT, which belongs to the class I methyltransferase family, while all other known HKMTs are class V methyltransferases that possess a conserved SET domain with a distinct 3-dimensional structural feature. In addition, DOT1L's substrate H3K79 is located in the ordered core structure of histone H3, while the substrates of all other HMTs are situated in the unordered histone tails.

Histone H3-lysine79 (H3K79) methyltransferase DOT1L plays critical roles in normal cell differentiation as well as initiation of acute leukemia. Thus potent inhibitors of DOT1L with low IC₅₀ values that are highly selective, and do not inhibit other methyltransferases, are particularly desirable to target acute leukemia. H3K79 methylation had also been found to be involved in breast cancer (BC).

Histone methyltransferase (HMT) DOT1L specifically methylates the residue Lys79 of histone H3 (H3K79), using S-adenosyl-L-methionine (SAM) as the enzyme cofactor, as seen in FIG. 1. Recent biological studies have demonstrated that DOT1L is a novel drug target for acute leukemia with MLL (mixed lineage leukemia) gene translocation. This subtype of leukemia accounts for ˜75% infant and ˜10% adult acute leukemia with a particularly poor prognosis. The majority of the translocated gene products, MLL-oncoproteins, are able to recruit DOT1L, which causes H3K79 hypermethylation leading to overexpression of leukemia relevant genes and eventually the cancer. Inhibitors of DOT1L should block the process and may potentially reverse the progress of MLL leukemia.

90% of BC deaths are due to metastasis or metastatic relapse. Breast cancer stem cells (BCSC), represent a small fraction of cells enriched in CD44⁺/CD24⁻ population that can initiate new tumors, and are responsible for metastasis and relapse. The clinical significance of BCSC is that because these cells proliferate slowly, they are intrinsically resistant to chemo-drugs that target rapidly dividing cancer cells, and thus are difficult to eliminate. These cells also show high epithelial mesenchymal transition (EMT) traits. EMT renders cancer cells to be more invasive, migratory and able to generate metastasis. EMT is characterized by overexpression of certain transcription factors such as Snail, Twist, Zeb1 and Zeb2. Inhibitors of DOT1L could target H3K79 methylation in BCSC, and thus circumvent the slow BCSC proliferation that cannot be addressed by drugs that target rapidly dividing cancer cells.

Previous medicinal chemistry studies have led to the discovery of several highly potent inhibitors of DOT1L with Kᵢ values of <1 nM, as representatively shown in FIG. 1. EPZ004777 (compound 1), the first disclosed DOT1L inhibitor, which can inhibit H3K79 methylation, lowers leukemia relevant gene expression, and induces differentiation of MLL leukemia cells, and thus further pharmacologically validated DOT1L to be a target for this type of leukemia. Structure based design was used to find N⁶-substituted SAH (S-adenosyl-L-homocysteine), such as compound 2, which are highly selective inhibitors of DOT1L. A mechanism based inhibitor design produced several aziridium analogs of SAM, such as compound 3 (FIG. 1) which almost quantitatively inactivates DOT1L. However, these compounds with a polar and charged amino acid moiety do not have cell activity, most likely due to limited cell membrane permeability. Efforts in identifying competitive DOT1L inhibitors resulted in the identification of compounds 4 and 5 with a similar structure and activity as compound 1. Structure activity relationship (SAR) investigations showed the urea group is critically important for the high potency, with each of the two NH moieties offering ˜25->50-fold activity enhancement. A crystallographic study revealed the binding structure of compound 1 in DOT1L. While the adenosine part of compound 1 adopts almost the same binding pose as that of SAM, DOT1L undergoes a large conformational change to favorably hold the tert-butylphenyl substituted urea group, with the —NHCONH— forming two hydrogen bonds with a sidechain of the enzyme.

A major problem with these ribose-containing inhibitors, e.g., compounds 1 and 4, is their metabolic instability, resulting in a short half-life in plasma. Compound 1 has to be infused continuously using a subcutaneously implanted osmotic pump to achieve a stable plasma drug concentration of ˜0.5 μM. This may be responsible for a relatively weak in vivo efficacy in prolonging the lifespan of the experimental animals in a mouse model of MLL translocated leukemia. As such, there exists a need for biologically active and metabolically stable DOT1L inhibitors.

### BRIEF DESCRIPTION OF THE DRAWINGS

For a more complete understanding of the present disclosure and advantages thereof, reference will now be made to the accompanying drawings/figures in which:

FIG. 1 illustrates a schematic of DOT1L catalyzed reaction and inhibitors;

FIG. 2 depicts compound SYC-522: with a Kᵢ=0.5 nM; compound EPZ004777 (1): with a Kᵢ of 0.3 nM; and S-(5′-adenosyl)-L-homocysteine (SAH), a non-selective compound: with a Kᵢ=160 nM;

FIG. 3A displays a fitted curve for compound of Formula VI by using Morrison tight binding model in Prism 5.0, from which a Kᵢ value was obtained;

FIG. 3B displays a fitted curve for compound of Formula VII by using Morrison tight binding model in Prism 5.0, from which a Kᵢ value was obtained;

FIG. 4 displays Western blots showing inhibition of H3K79 methylation in MV4-11 cells by compound 4 of FIG. 1, compound of Formula VI and compound of Formula VII;

FIG. 5A displays a graph of metabolic stability of DOT1L inhibitors in human liver microsome for compound 4 of FIG. 1, compound of Formula VI and compound of Formula VII;

FIG. 5B displays a graph of metabolic stability of DOT1L inhibitors in plasma for compound 4 of FIG. 1, compound of Formula VI and compound of Formula VII;

FIG. 6 displays structures of known DOT1L inhibitors and DOT1L inhibitors of the present disclosure;

FIG. 7A displays Western Blot images of the activity of SYC-522 and SYC-656 in histone methylation in MDA-MB231 cells;

FIG. 7B displays a graph of the activity of epigenetic inhibitors against mammosphere formation of MDA-MB231;

FIG. 7C displays a graph of the activity of SYC-522 on certain gene expression in MDA-MB231;

FIG. 7D displays graphs of tumor size in animal models over time during treatment with SYC-318 and SYC-656 versus control;

FIG. 8 displays structures of DOT1L inhibitors of the present disclosure; and

FIG. 9 displays a synthetic route for DOT1L inhibitors of the present disclosure.

### SUMMARY

Disclosed herein is a compound of Formula I, a pharmaceutically acceptable salt thereof, a prodrug thereof, or combinations thereof:

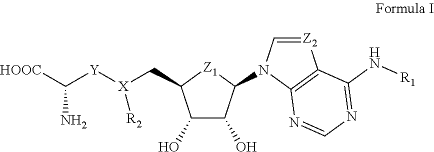

wherein R₁ is H, methyl, or benzyl;
   R₂ is 2-cyanoethyl, 2-methoxycarbonylethyl, or 2-iodoethyl;
   X is N or S; wherein if X=S, R₂=O;
   Y is C₃ or C₄;
   Z₁ is O, S, N, or CH₂; and
   Z₂ is N or CR₄, wherein R₄ is a halogen, alkyl, aryl or a 5- or 6-membered heterocycle; and
wherein said compound is selective for DOT1L Methyl Transferase.

Also disclosed herein is a compound of Formula II, a pharmaceutically acceptable salt thereof, a prodrug thereof, or combinations thereof:

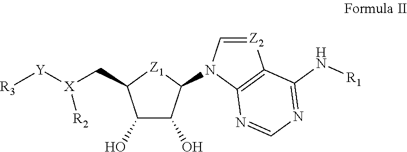

wherein R₁ is H, alkyl, or benzyl;
   R₂ is H, 2-cyanoethyl, 2-methoxycarbonylethyl, methyl, 2-iodoethyl, ethanol, butyl, or benzyl carbamate;
   X is N, C, or S; wherein if X=S, R₂=O; and wherein if X=C, R₂ is also equal to R₃ or R₁, and Y is also equal to R₁, R₂ or R₃;
   Y is C₁, C₂, C₃ or C₄;
   Z₁ is O, S, N, or CH₂;
   Z₂ is N, or CR₄, wherein R₄ is a halogen, alkyl, aryl or a 5- or 6-membered heterocycle;
   R₃ is H or selected from the following:

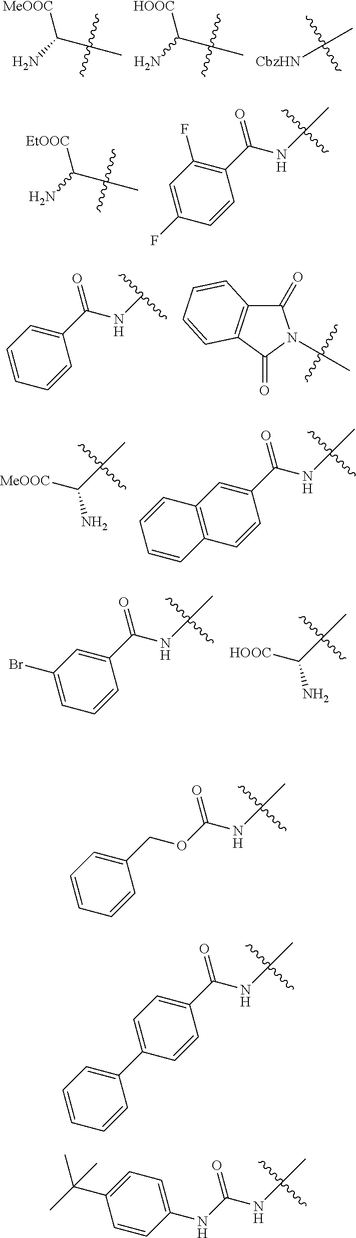

and wherein said compound is selective for DOT1L Methyl Transferase.

Further disclosed herein is a compound of Formula III, a pharmaceutically acceptable salt thereof, a prodrug thereof, or combinations thereof:

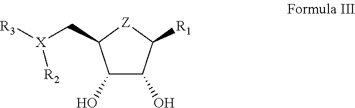

wherein R₁ is H, or a substituted or nonsubstituted: alkyl, cycloalkyl, morpholino, aryl, biaryl, fused biaryl, benzyl; heterocycle, purine, pyrimidine, alcohol, amine, amide, aldehyde, ketone, thiol; ester, ethers, carboxylate, acyl halide, imide, amidine, nitrile, cyano, thioaldehyde, ketone, thione, thioester, thioether, hydrazines, or disulphide;
   Z is CH₂;
   X is C, N, O or S; wherein if X=O, R₂=O;
   R₃ is H, O, or R₁;
   R₂ is H, O, or R₁;
   or R₃ and R₂ are cyclized together to form a substituted or nonsubstituted: alkyl, cycloalkyl, aryl, biaryl, fused biaryl, benzyl; heterocycle, purine, pyrimidine; and wherein said substituent is selected from R₁, R₂, R₃, X, halide; or combinations thereof; and
wherein said compound is selective for DOT1L Methyl Transferase.

Further disclosed herein is a compound of Formula IV, a pharmaceutically acceptable salt thereof, a prodrug thereof, or combinations thereof:

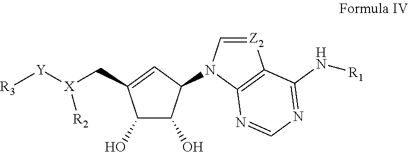

wherein R₁ is H, alkyl, or benzyl;
   R₂ is H, 2-cyanoethyl, 2-methoxycarbonylethyl, methyl, 2-iodoethyl, ethanol, butyl, or benzyl carbamate;
   X is N, C, or S; wherein if X=S, R₂=O; and wherein if X=C, R₂ is also equal to R₃ or R₁, and Y is also equal to R₁, R₂ or R₃;
   Y is C₁, C₂, C₃ or C₄;
   Z₂ is N, or CR₄, wherein R₄ can be a halogen, alkyl, aryl or a 5- or 6-membered heterocycle;
   R₃ is H or selected from the following:

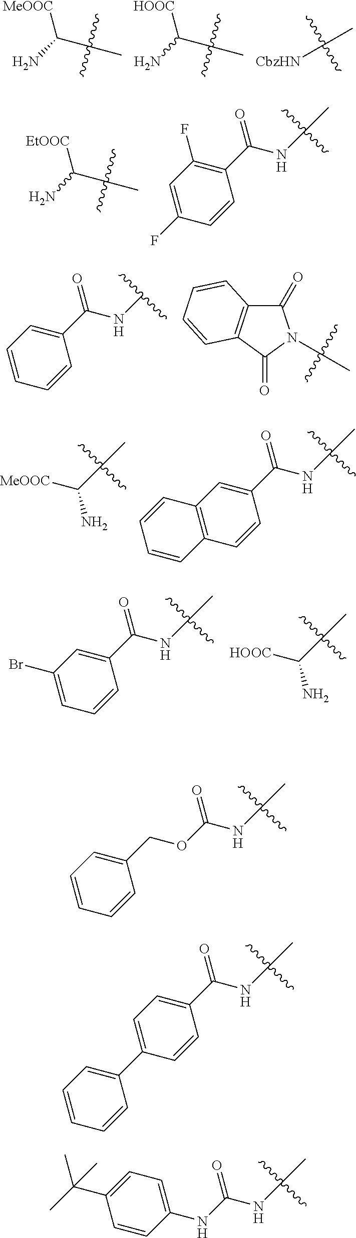

and wherein said compound is selective for DOT1L Methyl Transferase.

Further disclosed herein is a method of treating mixed lineage leukemia and/or breast cancer, wherein a compound of Formula VI, a pharmaceutically acceptable salt thereof, a prodrug thereof, or combinations thereof:

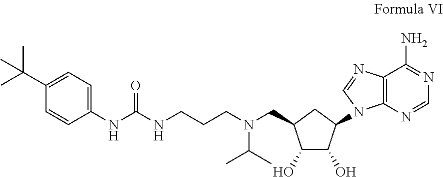 [SMILES: CC(C)N(CCCNC(=O)Nc1ccc(C(C)(C)C)cc1)C[C@H]1C[C@@H](n2cnc3c(N)ncnc32)[C@H](O)[C@@H]1O]

is administered to a subject, wherein said subject comprises cancer cells expressing a high level of DOT1L and wherein said compound selectively targets DOT1L.

Further disclosed herein is a method of treating mixed lineage leukemia, wherein a compound of Formula VII, a pharmaceutically acceptable salt thereof, a prodrug thereof, or combinations thereof:

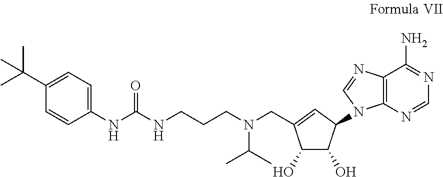 [SMILES: CC(C)N(CCCNC(=O)Nc1ccc(C(C)(C)C)cc1)CC1=C[C@@H](n2cnc3c(N)ncnc32)[C@H](O)[C@@H]1O]

is administered to a subject, wherein said subject comprises mixed lineage leukemia and wherein said compound selectively targets DOT1L.

Further disclosed herein is a method for the preparation of compounds of Formula II, pharmaceutically acceptable salts thereof, prodrugs thereof, or combinations thereof:

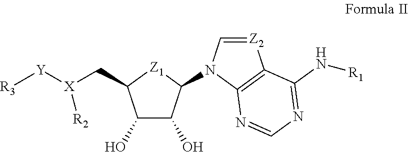

wherein R₁ is H, alkyl, or benzyl;
   R₂ is H, 2-cyanoethyl, 2-methoxycarbonylethyl, methyl, 2-iodoethyl, ethanol, butyl, or benzyl carbamate;
   X is N, C, or S; wherein if X=S, R₂=O; and wherein if X=C, R₂ is also equal to R₃ or R₁, and Y is also equal to R₁, R₂ or R₃;
   Y is C₁, C₂, C₃ or C₄;
   Z₁ is O, S, N, or CH₂;
   Z₂ is N, or CR₄, wherein R₄ is a halogen, alkyl, aryl or a 5- or 6-membered heterocycle;
   R₃ is H or selected from the following:

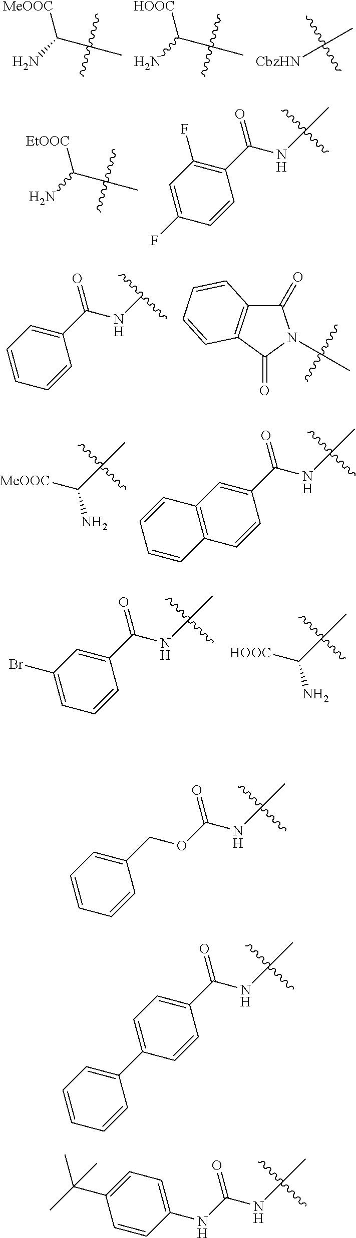

comprising reacting:

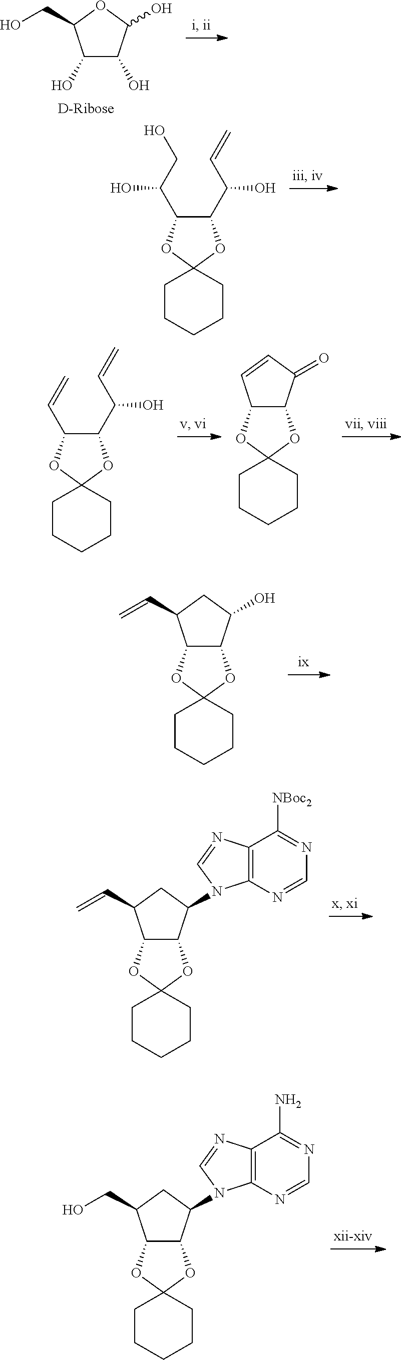

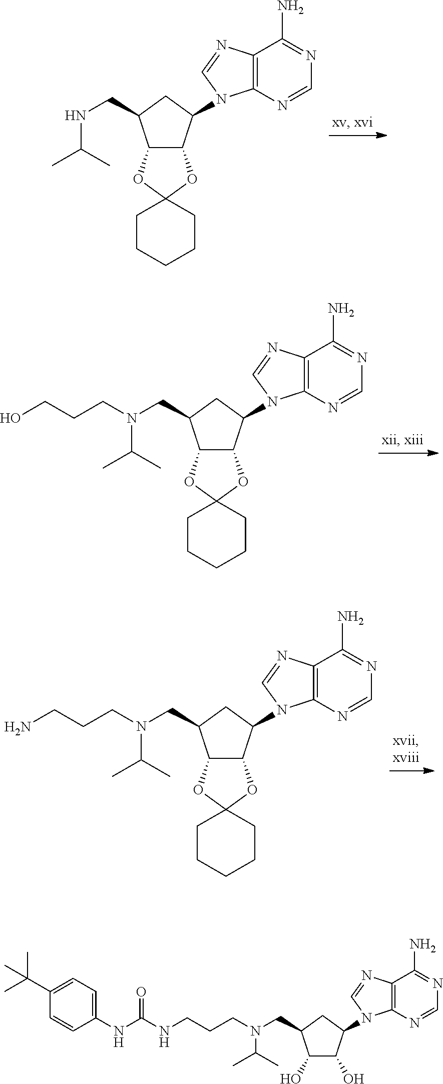 [SMILES: CC(C)NC[C@H]1C[C@@H](n2cnc3c(N)ncnc32)[C@@H]2OC3(CCCCC3)O[C@H]12 ; CC(C)N(CCCO)C[C@H]1C[C@@H](n2cnc3c(N)ncnc32)[C@@H]2OC3(CCCCC3)O[C@H]12 ; CC(C)N(CCCN)C[C@H]1C[C@@H](n2cnc3c(N)ncnc32)[C@@H]2OC3(CCCCC3)O[C@H]12 ; CC(C)N(CCCNC(=O)Nc1ccc(C(C)(C)C)cc1)C[C@H]1C[C@@H](n2cnc3c(N)ncnc32)[C@H](O)[C@@H]1O]

wherein: (i) cyclohexanone, cat. H₂SO₄; (ii) CH₂=CHMgBr, tetrahydrofuran (THF), −78° C.; (iii) NalO₄, MeOH/H₂O; (iv) Ph₃PCH₃Br, t-BuOK, THF; (v) 2^{nd} generation Grubbs' catalyst (5 mmol %), CH₂Cl₂; (vi) Dess-Martin periodinane (DMP), CH₂Cl₂; (vii) CH₂=CHMgBr, trimethylsilyl chloride (TMSCl), hexamethylphosphoramide, CuBr.Me₂S, THF, −78° C.; (viii) NaBH₄, CeCl₃.7H₂O, MeOH, 0° C.; (ix) 6,6-di-Boc-adenine, Ph₃P, diisopropyl azodicarboxylate (DIAD), THF; (x) O₃, CH₂Cl₂, −78° C., then NaBH₄/MeOH; (xi) trifluoroacetic acid, CH₂Cl₂; (xii) phthalimide, PPh₃, DIAD, THF; (xiii) NH₂NH₂, EtOH, 80° C.; (xiv) acetone, NaCNBH₃, MeOH; (xv) Methyl acrylate, MeOH, 65° C.; (xvi) LiAlH₄, THF, −15° C.; (xvii) 4-^{t}BuPhNCO, CH₂Cl₂; and (xviii) HCl, MeOH.

Further disclosed herein is a method for the preparation of compounds of Formula IV, pharmaceutically acceptable salts thereof, prodrugs thereof, or combinations thereof:

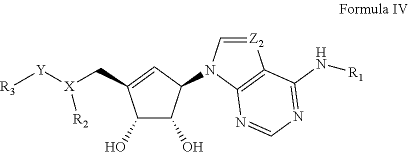

wherein
   R₁ is H, alkyl, or benzyl;
   R₂ is H, 2-cyanoethyl, 2-methoxycarbonylethyl, methyl, 2-iodoethyl, ethanol, butyl, or benzyl carbamate;
   X is N, C, or S; wherein if X=S, R₂=O; and wherein if X=C, R₂ is also equal to R₃ or R₁, and Y is also equal to R₁, R₂ or R₃;
   Y is C₁, C₂, C₃ or C₄;
   Z₂ is N, or CR₄, wherein R₄ can be a halogen, alkyl, aryl or a 5- or 6-membered heterocycle;
   R₃ is H or selected from the following:

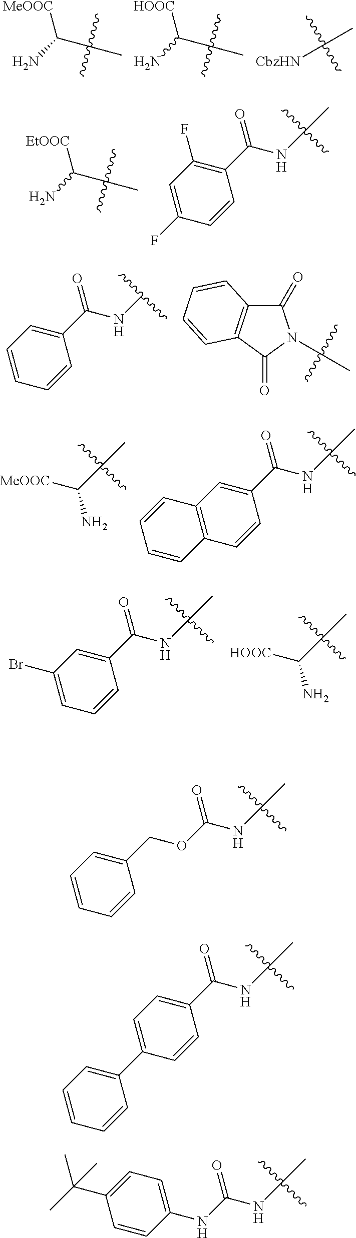

comprising reacting:

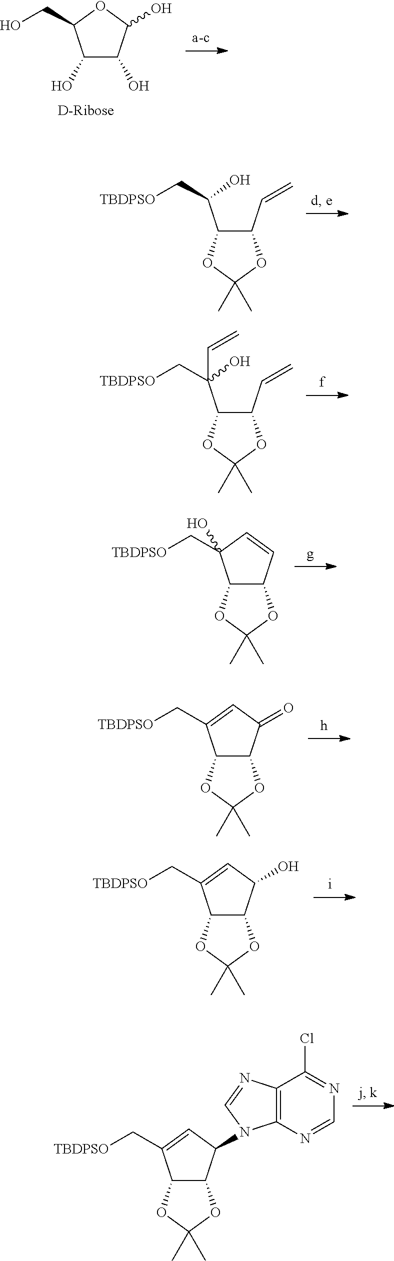

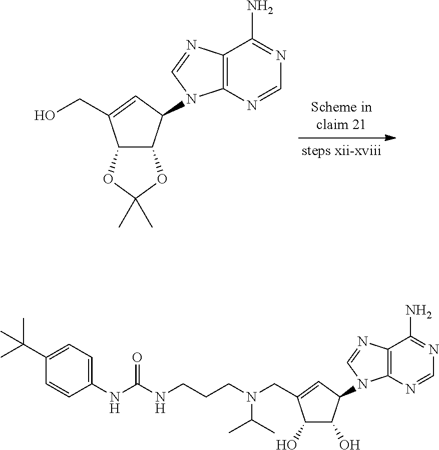 [SMILES: CC1(C)O[C@@H]2[C@H](O1)C(CO)=C[C@H]2n1cnc2c(N)ncnc21 ; CC(C)N(CCCNC(=O)Nc1ccc(C(C)(C)C)cc1)CC1=C[C@@H](n2cnc3c(N)ncnc32)[C@H](O)[C@@H]1O]

wherein: (a) acetone, cat. H₂SO₄; (b) tert-butyldiphenylsilyl chloride (TBDPSCl), Et₃N, 4-dimethylaminopyridine, dimethylformamide (DMF); (c) Ph₃PMeBr, t-BuOK, THF; (d) SO₃.Py, Et₃N, CH₂Cl₂; (e) CH₂=CHMgBr, THF, −78° C.; (f) 2^{nd} generation Grubbs catalyst (5 mmol %), CH₂Cl₂, reflux; (g) pyridinium dichromate (PDC), 4 Å molecular sieve, DMF; (h) NaBH₄, CeCl₃.7H₂O, MeOH, 0° C.; (i) 6-chloropurine, Ph₃P, DIAD, THF; (j) 7 M NH₃ in MeOH, 100° C.; (k) tetrabutylammonium fluoride, THF; and (1) H₂, 10% Pd/C, MeOH.

The foregoing has outlined rather broadly the features and technical advantages of the present invention in order that the detailed description of the invention that follows may be better understood. Additional features and advantages of the invention will be described hereinafter that form the subject of the claims of the invention. It should be appreciated by those skilled in the art that the conception and the specific embodiments disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims.

### DETAILED DESCRIPTION

It should be understood at the outset that although an illustrative implementation of one or more embodiments are provided below, the disclosed systems and/or methods may be implemented using any number of techniques, whether currently known or in existence. The disclosure should in no way be limited to the illustrative implementations, drawings, and techniques below, including the exemplary designs and implementations illustrated and described herein, but may be modified within the scope of the appended claims along with their full scope of equivalents.

The following discussion is directed to various exemplary embodiments of the invention. However, the embodiments disclosed should not be interpreted, or otherwise used, as limiting the scope of the disclosure, including the claims. In addition, one skilled in the art will understand that the following description has broad application, and the discussion of any embodiment is meant only to be exemplary of that embodiment, and that the scope of this disclosure, including the claims, is not limited to that embodiment.

Certain terms are used throughout the following description and claims to refer to particular features or components. As one skilled in the art will appreciate, different persons may refer to the same feature or component by different names. This document does not intend to distinguish between components or features that differ in name but not function. The drawing figures are not necessarily to scale. Certain features and components herein may be shown exaggerated in scale or in somewhat schematic form and some details of conventional elements may be omitted in interest of clarity and conciseness.

In the following discussion and in the claims, the terms “including” and “comprising” are used in an open-ended fashion, and thus should be interpreted to mean “including, but not limited to . . . . ” Also, the term “couple” or “couples” is intended to mean either an indirect or direct connection. Thus, if a first device couples to a second device, that connection may be through a direct engagement between the two devices, or through an indirect connection via other intermediate devices and connections. As used herein, the term “about,” when used in conjunction with a percentage or other numerical amount, means plus or minus 10% of that percentage or other numerical amount. For example, the term “about 80%,” would encompass 80% plus or minus 8%.

Abbreviations and Nomenclature: HKMT, histone lysine methyltransferases; PRMT, histone/protein arginine methyltransferases; H3K79, histone H3-lysine79; MLL, mixed lineage leukemia; SAM, S-(5′-adenosyl)-L-methionine; SAH, S-(5′-adenosyl)-L-homocysteine; BOC, tert-butoxycarbonyl; BC, breast cancer; BCSC, breast cancer stem cells.

Disclosed herein are embodiments of compositions for cancer treatment comprising DOT1L Methyl Transferase inhibitors, and methods of using the same. As will be appreciated by one of skill in the art, and with the help of this disclosure, DOT1L Methyl Transferase inhibitors are compounds that are selective for DOT1L Methyl Transferase. In an embodiment, a composition comprising a DOT1L Methyl Transferase inhibitor can be used for the treatment of mixed lineage leukemia (MLL) and/or breast cancer (BC). Although the DOT1L Methyl Transferase inhibitors disclosed herein will be discussed in detail in the context of MLL and/or BC, it should be understood that treatment for other types of cancer is also contemplated, wherein DOT1L Methyl Transferase inhibitors could be useful.

In an embodiment, the compositions comprising a DOT1L Methyl Transferase inhibitor can further comprise a carrier fluid. In an embodiment, the carrier fluids that may be used in the compositions comprising a DOT1L Methyl Transferase inhibitor include any carrier fluid suitable for chemotherapy. In an embodiment, the carrier fluid comprises a pharmaceutically acceptable carrier. For purposes of the disclosure herein, a “pharmaceutically acceptable carrier” is meant to encompass any carrier that does not interfere with effectiveness of a biological activity of any active ingredient (e.g., DOT1L Methyl Transferase inhibitor) and that is not toxic to an individual to which it is administered. “Pharmaceutically acceptable” as used herein adheres to the U.S. Food and Drug Administration guidelines.

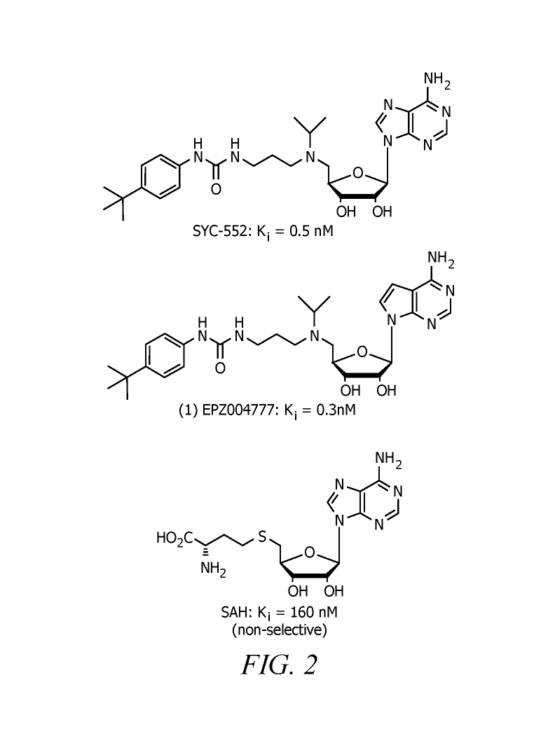

DOT1L is recruited by onco-MLL fusion partners AF4/9/10/ENL, hypermethylate H3K79, cause overexpression of leukemia relevant genes and eventually lead to leukemia. In addition, we, as well as others, have (independently) discovered very potent DOT1L inhibitors (e.g., SYC-522 and EPZ004777 shown in FIG. 2) with Kᵢ as low as 0.3 nM. These compounds can inhibit H3K79 methylation, downregulate H3K79 targeted oncogene expressions, induce differentiation of cancer stem cells (CSCs), and eventually block the proliferation of MLL leukemia cells. Of particular interest is that these compounds have no general cytotoxicity (EC50>100 μM against non-MLL leukemia cells), in contrast to traditional chemotherapeutics. These results show DOT1L inhibitors have the potential to become the first targeted therapeutics for MLL-leukemia.

Referring to FIG. 2, SYC-522 had been found herein to have selective activity against the proliferation of claudin-low breast cancer (CLBC) cells such as MDA-MB231, while it is weakly active or inactive against basal-like triple-negative breast cancer (TNBC) as well as other cancerous and normal cells.

SYC-522 (FIG. 2) also inhibits the proliferation of CLBC stem cells. Mechanistically, SYC-522 specifically blocks H3K79 methylation, inhibits the self-renewal ability and induces differentiation of breast CSC, and reverses certain dysregulated gene expression of claudins, E-cadherin and EMT in MDA-MB231. Two histone methylation inhibitors were found to be able to completely inhibit the in vivo growth of MDA-MB231 xenograft mouse model.

H3K79 methylation is a drug target for CLBC. Rational inhibitor design and medicinal chemistry was herein used to develop two series of H3K79 methylation inhibitors that have selective activity and good pharmacokinetics (PK) and toxicological (PK/Tox) properties. The enzyme and cell activity of such compounds have been assessed to be good drug candidates in two CLBC mouse models. The first was the MDA-MB231 xenograft model, and the second was an aggressive, metastatic MDA-MB231 model which can produce lung metastasis in <5 weeks after transplantation. Compounds that can inhibit the lung metastatic tumors may be promising drug candidates for CLBC. Thus using a specific DOT1L inhibitor as a probe in the embodiments described herein shows that DOT1L/H3K79 methylation is a drug target for CLBC, which is characterized by early metastasis with a particularly poor prognosis, and embodiments described herein show that H3K79 methylation inhibitors have selective activity against CLBC, representing the potential to be the first targeted therapeutics for this subtype of breast cancer. Embodiments described herein, also provide a highly aggressive, metastatic CLBC mouse model that can be used to evaluate the ability of a drug to inhibit breast cancer metastasis. Further embodiments provide for novel, metabolically stable H3K79 methylation inhibitors that are good drug candidates for in vivo preclinical studies as well as possible future clinical trials.

DOT1L catalyzes the methylation reaction of the δ-amino group of H3K79 up to trimethylation (H3K79Me3) using S-adenosyl-L-methionine (SAM) as the enzyme cofactor, producing the methylated substrate and S-adenosyl-L-homocysteine (SAH). SAH (FIG. 2) was found to be a strong inhibitor of DOT1L with a Kᵢ of 160 nM. However, in addition to limited cell membrane permeability, SAH has a broad inhibitory activity against many other histone methyltransferases (HMTs). Selective DOT1L inhibitors are highly desirable in the context of drug discovery.

In an embodiment, a DOT1L Methyl Transferase inhibitor can comprise a compound of Formula I, a pharmaceutically acceptable salt thereof, a prodrug thereof, or combinations thereof:

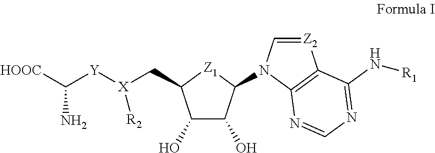

wherein R₁ can be H, methyl, or benzyl; R₂ can be 2-cyanoethyl, 2-methoxycarbonylethyl, or 2-iodoethyl; X can be N or S; wherein if X=S, R₂=O; Y can be C₃ or C₄, Z₁ can be O, S, N, or CH₂, and Z₂ can be N, or CR₄, wherein R₄ is a halogen, alkyl, aryl or a 5- or 6-membered heterocycle; and wherein said compound can be selective for DOT1L Methyl Transferase. For purposes of the disclosure herein, a “pharmaceutically acceptable salt” is meant to encompass any salt of the DOT1L Methyl Transferase inhibitor that does not interfere with effectiveness of any other active ingredients (e.g., DOT1L Methyl Transferase inhibitor) of a treatment composition and that is not toxic to an individual to which it is administered. “Pharmaceutically acceptable” as used herein adheres to the U.S. Food and Drug Administration guidelines. Further, for purposes of the disclosure herein, a “prodrug” is meant to encompass any precursor of DOT1L Methyl Transferase inhibitor that is administered as medication in an inactive or less than fully active form (e.g., a prodrug of a DOT1L Methyl Transferase inhibitor), and then it becomes converted to its active form (e.g., DOT1L Methyl Transferase inhibitor) through a normal metabolic process, such as for example hydrolysis of an ester form of the drug.

In an embodiment, a DOT1L Methyl Transferase inhibitor can comprise a compound of Formula II, a pharmaceutically acceptable salt thereof, a prodrug thereof, or combinations thereof:

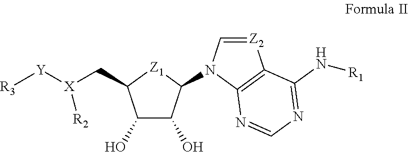

wherein R₁ can be H, alkyl, or benzyl; R₂ can be H, 2-cyanoethyl, 2-methoxycarbonylethyl, methyl, 2-iodoethyl, ethanol, butyl, or benzyl carbamate; X can be N, C, or S; wherein if X=S, R₂=O; and wherein if X=C, R₂ can also be equal to R₃ or R₁, and Y can also be equal to R₁, R₂ or R₃; Y can be C₁, C₂, C₃ or C₄; Z₁ can be 0, S, N, or CH₂; Z₂ can be N, or CR₄, wherein R₄ can be a halogen, alkyl, aryl or a 5- or 6-membered heterocycle; R₃ can be H or selected from the following:

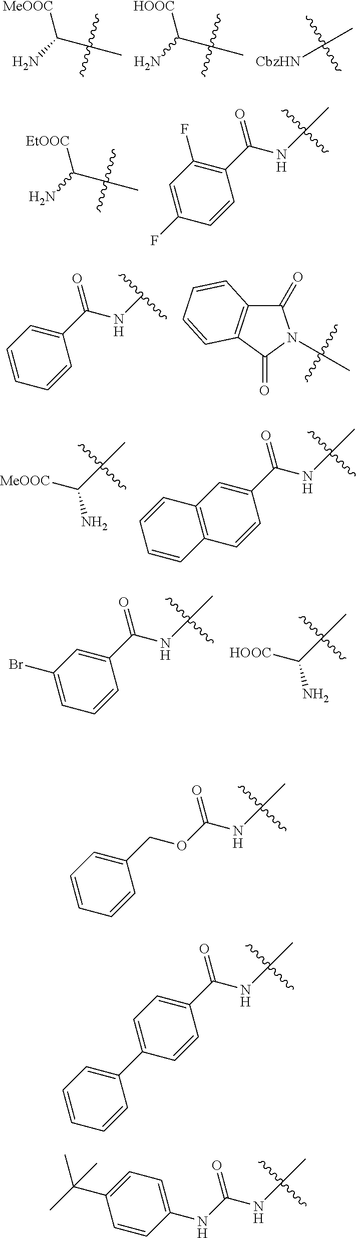

and wherein said compound can be selective for DOT1L Methyl Transferase.

In an embodiment, a DOT1L Methyl Transferase inhibitor can comprise a compound of Formula III, a pharmaceutically acceptable salt thereof, a prodrug thereof, or combinations thereof:

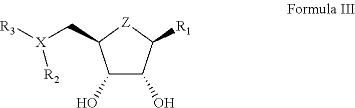

wherein R₁ can be H, or a substituted or nonsubstituted: alkyl, cycloalkyl, morpholino, aryl, biaryl, fused biaryl, benzyl; heterocycle, purine, pyrimidine, alcohol, amine, amide, aldehyde, ketone, thiol; ester, ethers, carboxylate, acyl halide, imide, amidine, nitrile, cyano, thioaldehyde, ketone, thione, thioester, thioether, hydrazines, or disulphide; Z can be CH₂; X can be C, N, O or S; wherein if X=O, R₂=O; R₃ can be H, O, or R₁; R₂ can be H, O, or R₁; or R₃ and R₂ can be cyclized together to form a substituted or nonsubstituted: alkyl, cycloalkyl, aryl, biaryl, fused biaryl, benzyl; heterocycle, purine, pyrimidine; and wherein said substituent may be selected from R₁, R₂, R₃, X, halide; or combinations thereof; and wherein said compound can be selective for DOT1L Methyl Transferase.

In an embodiment, a DOT1L Methyl Transferase inhibitor can comprise a compound of Formula IV, a pharmaceutically acceptable salt thereof, a prodrug thereof, or combinations thereof:

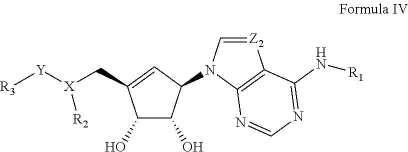

wherein R₁ can be H, alkyl, or benzyl; R₂ can be H, 2-cyanoethyl, 2-methoxycarbonylethyl, methyl, 2-iodoethyl, ethanol, butyl, or benzyl carbamate; X can be N, C, or S; wherein if X=S, R₂=O; and wherein if X=C, R₂ can also be equal to R₃ or R₁, and Y can also be equal to R₁, R₂ or R₃; Y can be C₁, C₂, C₃ or C₄; Z₂ can be N, or CR₄, wherein R₄ can be a halogen, alkyl, aryl or a 5- or 6-membered heterocycle; R₃ can be H or selected from the following:

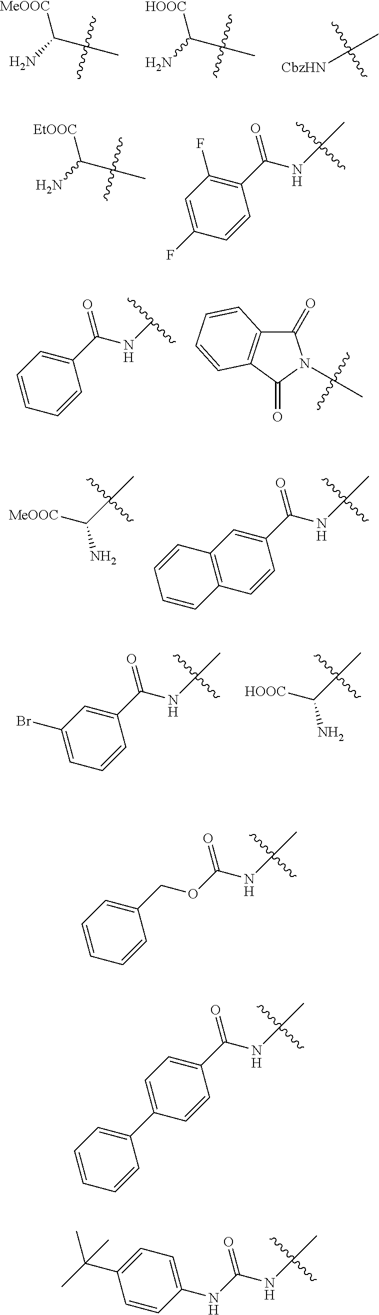

and wherein said compound can be selective for DOT1L Methyl Transferase.

In an embodiment, a DOT1L Methyl Transferase inhibitor can comprise a compound of Formula V, a pharmaceutically acceptable salt thereof, a prodrug thereof, or combinations thereof:

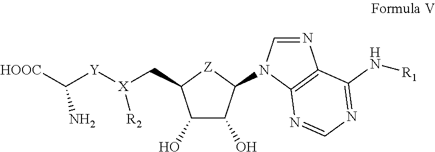

wherein R₁ can be H, methyl, or benzyl; R₂ can be 2-cyanoethyl, 2-methoxycarbonylethyl, or 2-iodoethyl; X can be N or S; wherein if X=S, R₂=O; Y can be C₃ or C₄, and Z can be O, S, N, or CH₂; and wherein said compound can be selective for DOT1L Methyl Transferase. In an embodiment, the compound of Formula I comprises the compound of Formula V.

In an embodiment, a DOT1L Methyl Transferase inhibitor as disclosed herein, such as a compound of Formula I, a compound of Formula II, a compound of Formula III, and/or a compound of Formula IV, can be provided wherein R₁ can specifically bind in the hydrophobic pocket comprising Phe223, Leu224, Val249, Lys187 and Pro133 of the DOT1L protein, thereby selectively inhibiting DOT1L Methyl Transferase activity.

In another embodiment, a DOT1L Methyl Transferase inhibitor as disclosed herein, such as a compound of Formula I, a compound of Formula II, a compound of Formula III, and/or a compound of Formula IV, can be provided wherein the N6 hydrogen forms a hydrogen bond with Asp222 of the DOT1L protein, thereby selectively inhibiting DOT1L Methyl Transferase activity.

In yet another embodiment, a DOT1L Methyl Transferase inhibitor as disclosed herein, such as a compound of Formula I, a compound of Formula II, a compound of Formula III, and/or a compound of Formula IV, can be provided wherein said compound can have specificity for DOT1L and can be substantially free of specificity for CARM1, PRMT1, G9a and SUV39H1 Methyl Transferases.

In an embodiment, a DOT1L Methyl Transferase inhibitor can comprise a compound of Formula VI, a pharmaceutically acceptable salt thereof, a prodrug thereof, or combinations thereof:

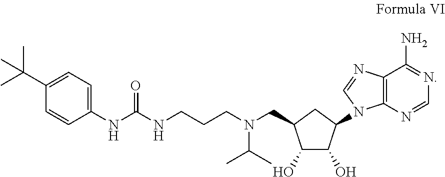 [SMILES: CC(C)N(CCCNC(=O)Nc1ccc(C(C)(C)C)cc1)C[C@H]1C[C@@H](n2cnc3c(N)ncnc32)[C@H](O)[C@@H]1O]

In an embodiment, a DOT1L Methyl Transferase inhibitor can comprise a compound of Formula VII, a pharmaceutically acceptable salt thereof, a prodrug thereof, or combinations thereof:

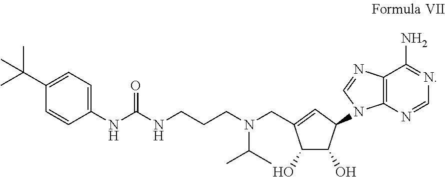 [SMILES: CC(C)N(CCCNC(=O)Nc1ccc(C(C)(C)C)cc1)CC1=C[C@@H](n2cnc3c(N)ncnc32)[C@H](O)[C@@H]1O]

In an embodiment, a method for the preparation of compounds of general Formula II, pharmaceutically acceptable salts thereof, prodrugs thereof, or combinations thereof:

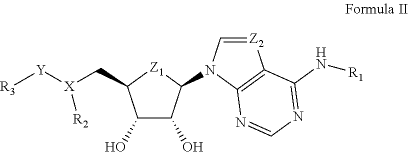

wherein R₁ can be H, alkyl, or benzyl; R₂ can be H, 2-cyanoethyl, 2-methoxycarbonylethyl, methyl, 2-iodoethyl, ethanol, butyl, or benzyl carbamate; X can be N, C, or S; wherein if X=S, R₂=O; and wherein if X=C, R₂ can also be equal to R₃ or R₁, and Y can also be equal to R₁, R₂ or R₃; Y can be C₁, C₂, C₃ or C₄; Z₁ can be 0, S, N, or CH₂; Z₂ can be N, or CR₄, wherein R₄ can be a halogen, alkyl, aryl or a 5- or 6-membered heterocycle; R₃ can be H or selected from the following:

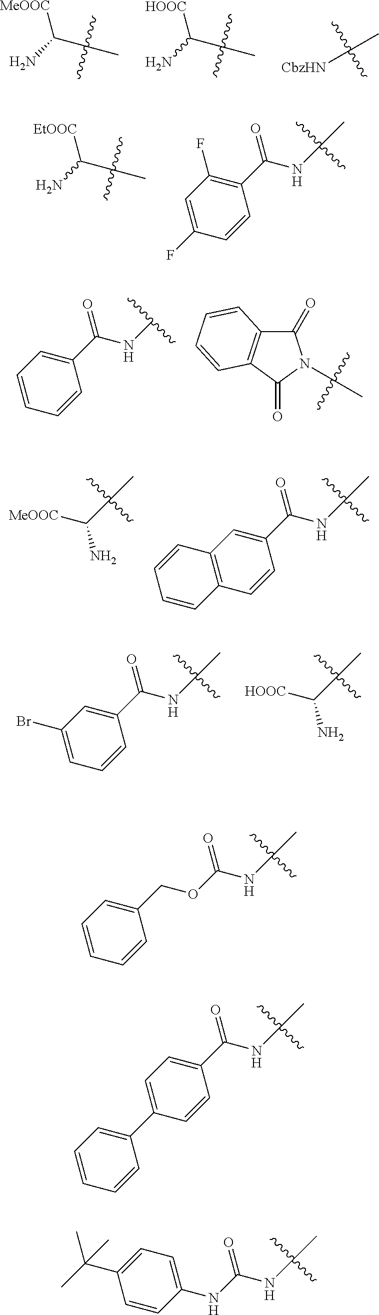

can comprise reacting:

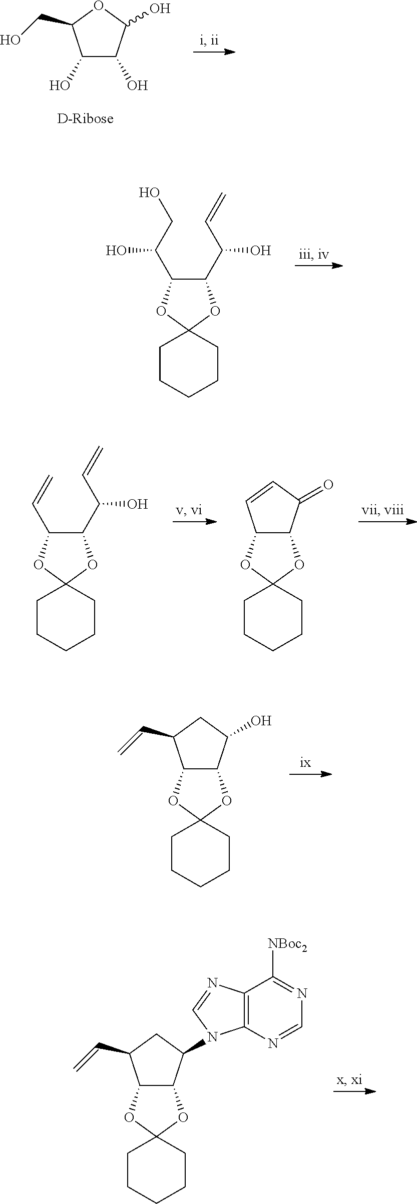

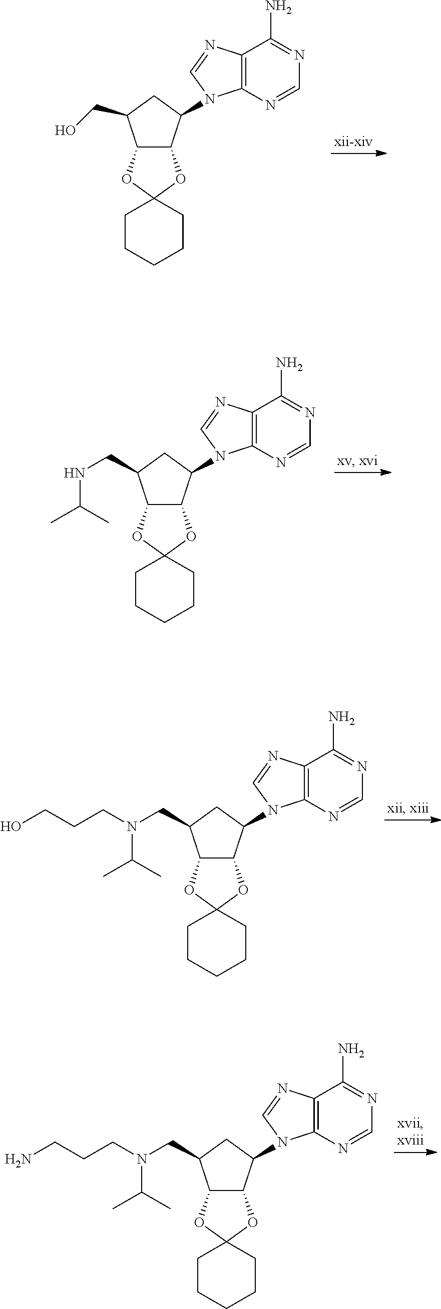 [SMILES: Nc1ncnc2c1ncn2[C@@H]1C[C@H](CO)[C@H]2OC3(CCCCC3)O[C@H]21 ; CC(C)NC[C@H]1C[C@@H](n2cnc3c(N)ncnc32)[C@@H]2OC3(CCCCC3)O[C@H]12 ; CC(C)N(CCCO)C[C@H]1C[C@@H](n2cnc3c(N)ncnc32)[C@@H]2OC3(CCCCC3)O[C@H]12 ; CC(C)N(CCCN)C[C@H]1C[C@@H](n2cnc3c(N)ncnc32)[C@@H]2OC3(CCCCC3)O[C@H]12]

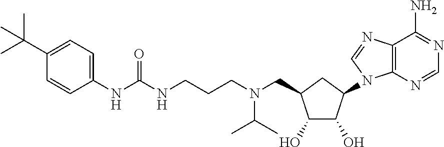 [SMILES: CC(C)N(CCCNC(=O)Nc1ccc(C(C)(C)C)cc1)C[C@H]1C[C@@H](n2cnc3c(N)ncnc32)[C@H](O)[C@@H]1O]

wherein: (i) cyclohexanone, cat. H₂SO₄; (ii) CH₂=CHMgBr, tetrahydrofuran (THF), −78° C.; (iii) NalO₄, MeOH/H₂O; (iv) Ph₃PCH₃Br, t-BuOK, THF; (v) 2^{nd} generation Grubbs' catalyst (5 mmol %), CH₂Cl₂; (vi) Dess-Martin periodinane (DMP), CH₂Cl₂; (vii) CH₂=CHMgBr, trimethylsilyl chloride (TMSCl), hexamethylphosphor-amide, CuBr.Me₂S, THF, −78° C.; (viii) NaBH₄, CeCl₃.7H₂O, MeOH, 0° C.; (ix) 6,6-di-Boc-adenine, Ph₃P, diisopropyl azodicarboxylate (DIAD), THF; (x) O₃, CH₂Cl₂, −78° C., then NaBH₄/MeOH; (xi) trifluoroacetic acid, CH₂Cl₂; (xii) phthalimide, PPh₃, DIAD, THF; (xiii) NH₂NH₂, EtOH, 80° C.; (xiv) acetone, NaCNBH₃, MeOH; (xv) Methyl acrylate, MeOH, 65° C.; (xvi) LiAlH₄, THF, −15° C.; (xvii) 4-^{t}BuPhNCO, CH₂Cl₂; and (xviii) HCl, MeOH.

In an embodiment, a method for the preparation of compounds of general Formula IV, pharmaceutically acceptable salts thereof, prodrugs thereof, or combinations thereof:

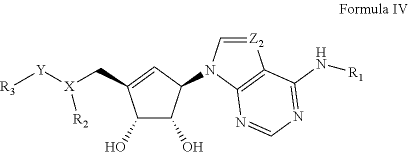

wherein R₁ can be H, alkyl, or benzyl; R₂ can be H, 2-cyanoethyl, 2-methoxycarbonylethyl, methyl, 2-iodoethyl, ethanol, butyl, or benzyl carbamate; X can be N, C, or S; wherein if X=S, R₂=O; and wherein if X=C, R₂ can also be equal to R₃ or R₁, and Y can also be equal to R₁, R₂ or R₃; Y can be C₁, C₂, C₃ or C₄; Z₂ can be N, or CR₄, wherein R₄ can be a halogen, alkyl, aryl or a 5- or 6-membered heterocycle; R₃ can be H or selected from the following:

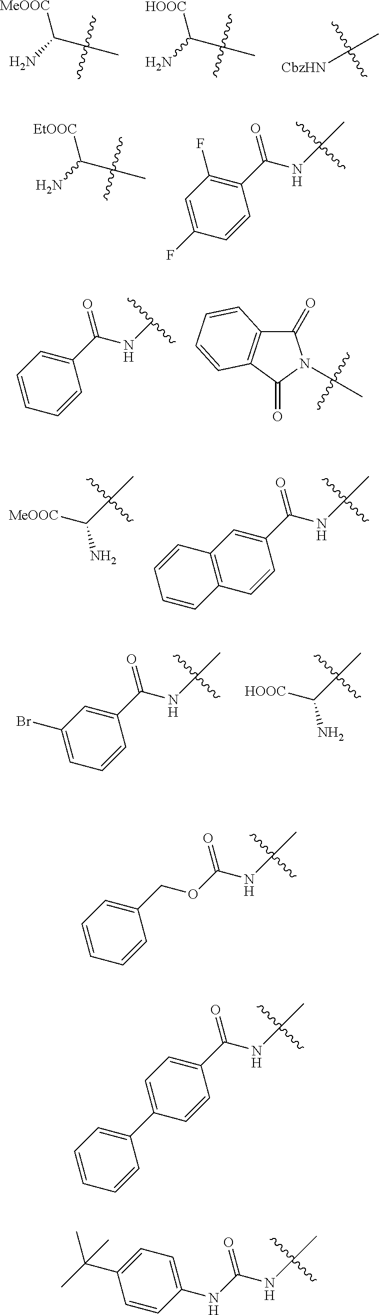

can comprise reacting:

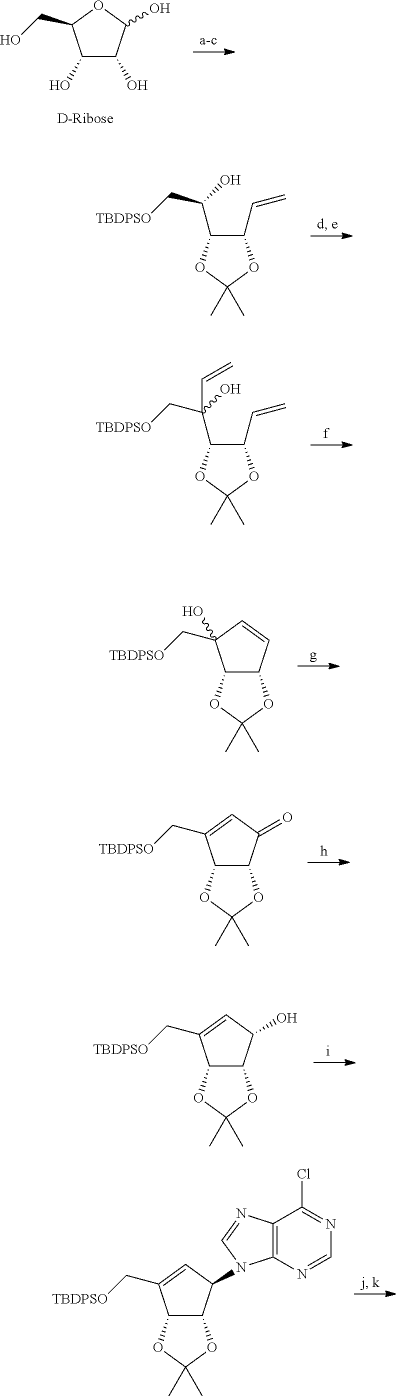

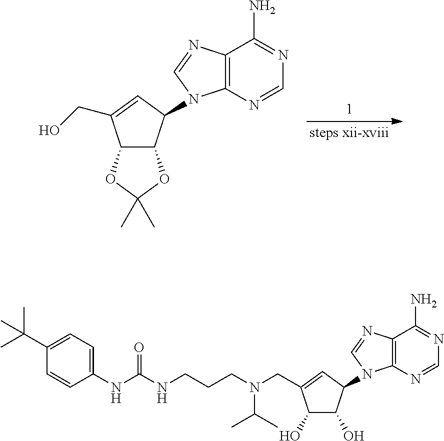 [SMILES: CC1(C)O[C@@H]2[C@H](O1)C(CO)=C[C@H]2n1cnc2c(N)ncnc21 ; CC(C)N(CCCNC(=O)Nc1ccc(C(C)(C)C)cc1)CC1=C[C@@H](n2cnc3c(N)ncnc32)[C@H](O)[C@@H]1O]

wherein: (a) acetone, cat. H₂SO₄; (b) tert-butyldiphenylsilyl chloride (TBDPSCl), Et₃N, 4-dimethylaminopyridine, dimethylformamide (DMF); (c) Ph₃PMeBr, t-BuOK, THF; (d) SO₃.Py, Et₃N, CH₂Cl₂; (e) CH₂=CHMgBr, THF, −78° C.; (f) 2^{nd} generation Grubbs catalyst (5 mmol %), CH₂Cl₂, reflux; (g) pyridinium dichromate (PDC), 4 Å molecular sieve, DMF; (h) NaBH₄, CeCl₃.7H₂O, MeOH, 0° C.; (i) 6-chloropurine, Ph₃P, DIAD, THF; (j) 7 M NH₃ in MeOH, 100° C.; (k) tetrabutylammonium fluoride, THF; and (1) H₂, 10% Pd/C, MeOH, wherein step (I) of preparing a compound of Formula IV corresponds to steps xii-xvii as previously described herein for the preparation of the compound of Formula II.

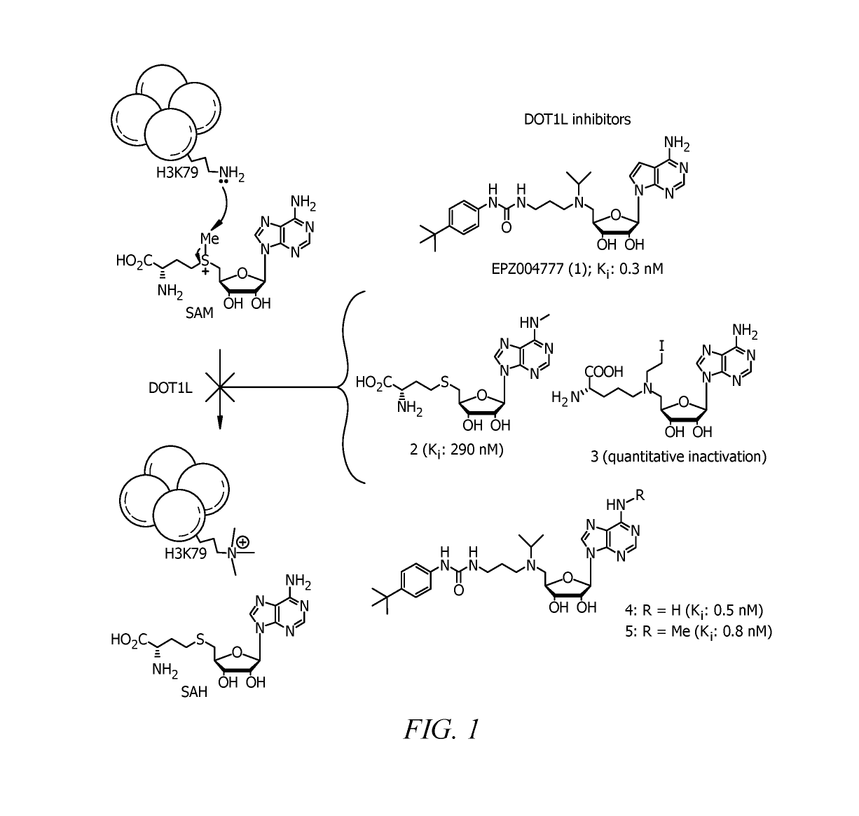

As will be appreciated by one of skill in the art, and with the help of this disclosure, adenosine or deaza-adenosine moiety can be recognized by many enzymes, thereby leading to a rapid cleavage of adenine and/or 5′-substituent. In an embodiment, a method for the preparation of the a DOT1L Methyl Transferase inhibitors can comprise replacing the metabolically labile ribose (i.e., the ribofuranose) group in compounds 1 and 4 in FIG. 1 with a cyclopentane ring to yield the compound of Formula VI or with a cyclopentene ring to yield the compound of Formula VII:

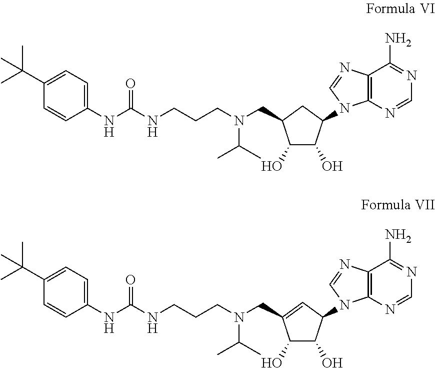

Methods of synthesis for each class of compound was developed, as described in the following reaction schemes for the synthesis of the compound of Formula VI and for the synthesis of the compound of Formula VII, respectively.

In an embodiment, the compound of Formula VI can be synthesized by following a 20-step synthesis of as shown in Reaction Scheme I, starting from readily available D-ribose, with the key steps being to construct the central cyclopentane ring with the correct stereochemistry of its substituents:

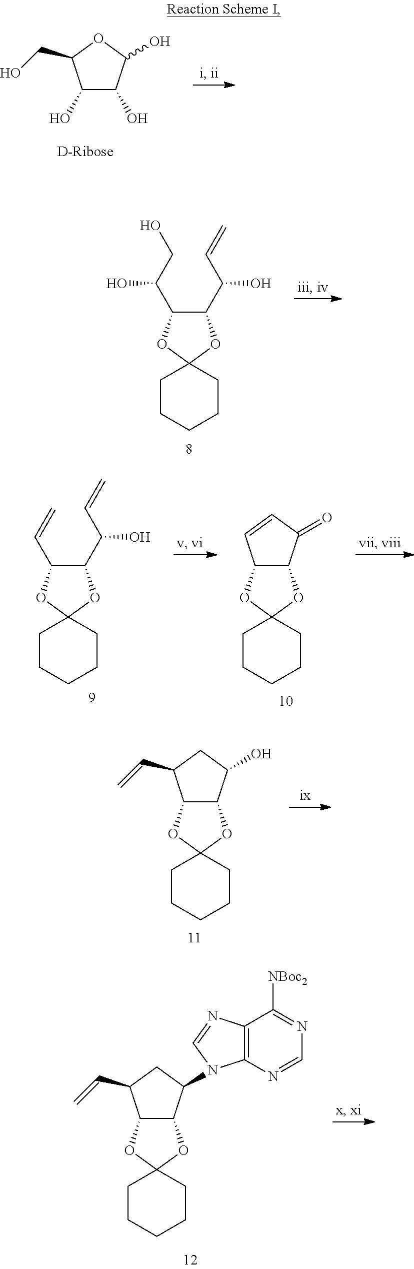

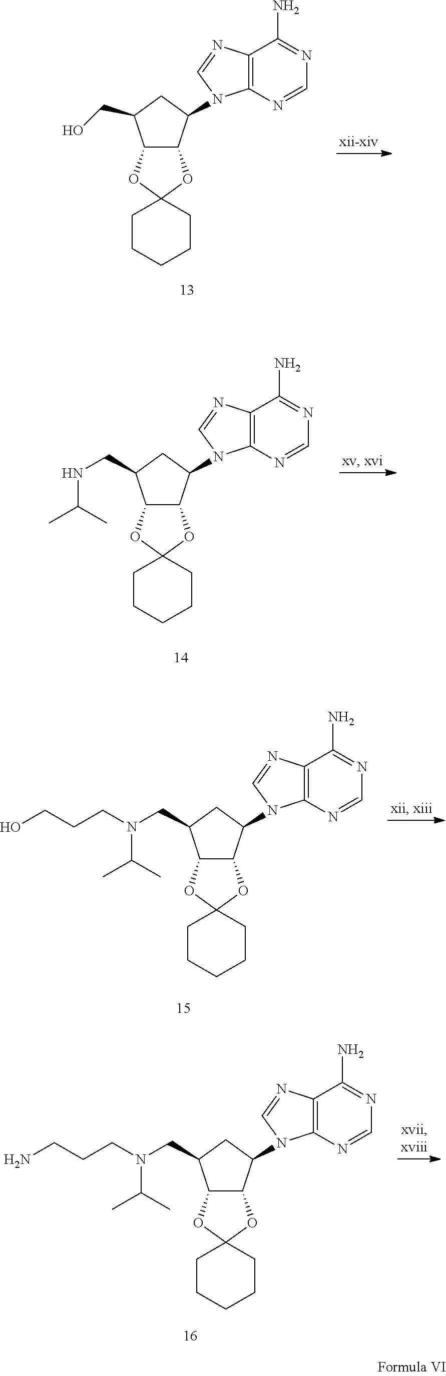 [SMILES: Nc1ncnc2c1ncn2[C@@H]1C[C@H](CO)[C@H]2OC3(CCCCC3)O[C@H]21 ; CC(C)NC[C@H]1C[C@@H](n2cnc3c(N)ncnc32)[C@@H]2OC3(CCCCC3)O[C@H]12 ; CC(C)N(CCCO)C[C@H]1C[C@@H](n2cnc3c(N)ncnc32)[C@@H]2OC3(CCCCC3)O[C@H]12 ; CC(C)N(CCCN)C[C@H]1C[C@@H](n2cnc3c(N)ncnc32)[C@@H]2OC3(CCCCC3)O[C@H]12]

wherein reagents and conditions can be: (i) cyclohexanone, cat. H₂SO₄; (ii) CH₂=CHMgBr, THF, −78° C., 70% for 2 steps; (iii) NalO₄, MeOH/H₂O; (iv) Ph₃PCH₃Br, t-BuOK, THF, 87% for two steps; (v) 2^{nd} generation Grubbs' catalyst (5 mmol %), CH₂Cl₂; (vi) DMP, CH₂Cl₂, 86% for two steps; (vii) CH₂=CHMgBr, TMSCl, hexamethylphosphor-amide, CuBr.Me₂S, THF, −78° C., 89%; (viii) NaBH₄, CeCl₃.7H₂O, MeOH, 0° C., 96%; (ix) 6,6-di-Boc-adenine, Ph₃P, DIAD, THF, 73%; (x) O₃, CH₂Cl₂, −78° C., then NaBH₄/MeOH, 92%; (xi) trifluoroacetic acid, CH₂Cl₂, 96%; (xii) phthalimide, PPh₃, DIAD, THF, 92%; (xiii) NH₂NH₂, EtOH, 80° C., 99%; (xiv) acetone, NaCNBH₃, MeOH, 95%; (xv) Methyl acrylate, MeOH, 65° C., 99%; (xvi) LiAlH₄, THF, −15° C., 90%; (xvii) 4-^{t}BuPhNCO, CH₂Cl₂, 95%; and (xviii) HCl, MeOH, 92%.

In an embodiment, the 2′,3′-dihydroxyls of D-ribose can be selectively protected with cyclohexanone and the product can be treated with vinylmagnesium bromide to give vinyl-substituted ribose derivative 8. Its vicinal 4′,5′-diol can be oxidatively cleaved by NalO₄ and the resulting 4′-CH═O treated with CH₂=PPh₃ to afford diene 9, which can be subjected to a ring-closing metathesis reaction, followed by oxidation using Dess-Martin periodinane (DMP) to produce cyclopentenone 10. Due to a high stereoselectivity rendered by the bicyclic ring of 10, nucleophilic attacks preferably occur from the less hindered up side of the cyclopentane ring. Thus, 1,4-addition of a vinyl group to 10 and the ensuing NaBH₄-mediated reduction can give exclusively the key cyclopentane intermediate 11. A Mitsunobu reaction using di-BOC (tert-butyloxycarbonyl) protected adenine with 11 can afford compound 12, which can be treated with O₃ at −78° C. followed by NaBH₄ to give 13 with a 5′-OH (according to the corresponding ribofuranose nomenclature). Using a Mitsunobu reaction with phthalimide followed by treatment with NH₂NH₂, the 5′-OH of 13 can be converted to an —NH₂, which can then be mono-substituted with an isopropyl group using acetone/NaCNBH₃ to produce compound 14. Conjugated addition of 14 to methyl acrylate followed by LiAlH₄ reduction can give compound 15. Its —OH can again be converted to a primary —NH2, affording compound 16, which can be treated with 4-tert-butylphenyl isocyanate followed by deprotection of 2′, 3′-hydroxyls to give the final product characterized by Formula VI. In an embodiment, the yield of the compound of Formula VI from D-ribose can be greater than about 10 wt. %, alternatively greater than about 20 wt. %, or alternatively greater than about 30 wt. %, based on the theoretical amount of compound of Formula VI that could be obtained from D-ribose by following the indicated reaction scheme.

In an embodiment, the compound of Formula VII, as well as epi-6, a diastereomer of the compound of Formula VI that can be used to determine the importance of the stereochemistry at 4′-position, can be synthesized as shown in Reaction Scheme II:

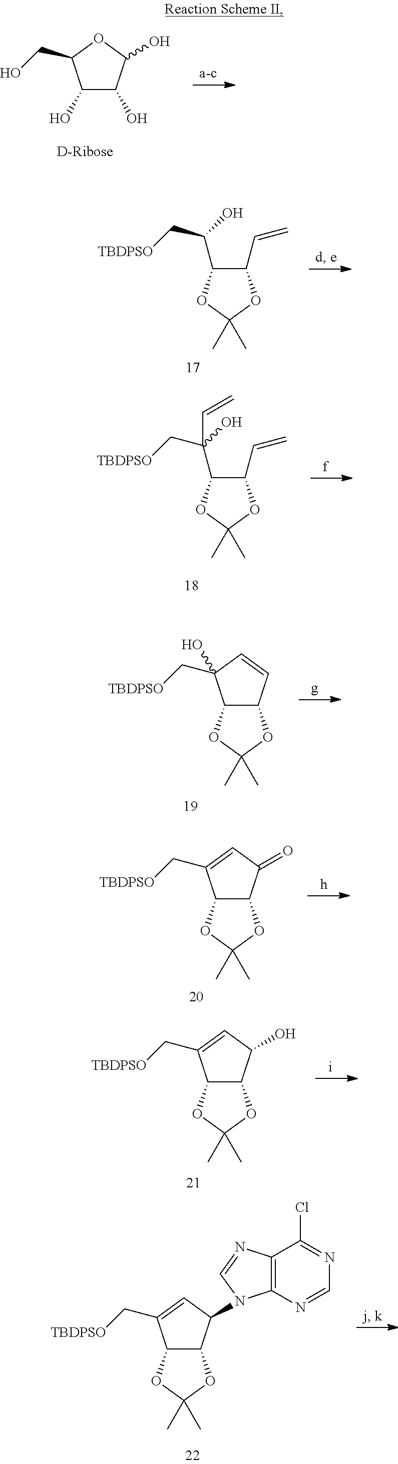

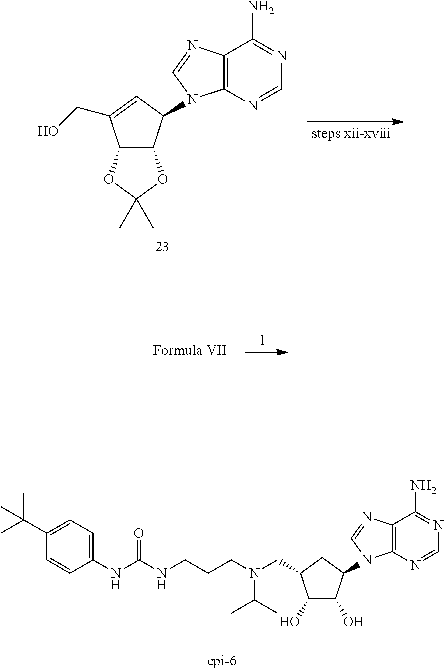 [SMILES: CC1(C)O[C@@H]2[C@H](O1)C(CO)=C[C@H]2n1cnc2c(N)ncnc21 ; CC(C)N(CCCNC(=O)Nc1ccc(C(C)(C)C)cc1)C[C@@H]1C[C@@H](n2cnc3c(N)ncnc32)[C@H](O)[C@@H]1O]

wherein reagents and conditions can be: (a) acetone, cat. H₂SO₄, 85%; (b) TBDPSCl, Et₃N, 4-dimethylaminopyridine, DMF, 98%; (c) Ph₃PMeBr, t-BuOK, THF, 92%; (d) SO₃.Py, Et₃N, CH₂Cl₂, 97%; (e) CH₂=CHMgBr, THF, −78° C., 93%; (f) 2^{nd} generation Grubbs catalyst (5 mmol %), CH₂Cl₂, reflux, 95%; (g) PDC, 4 Å molecular sieve, DMF, 87%; (h) NaBH₄, CeCl₃.7H₂O, MeOH, 0° C., 98%; (i) 6-chloropurine, Ph₃P, DIAD, THF, 95%; (j) 7 M NH₃ in MeOH, 100° C.; (k) tetrabutylammonium fluoride, THF, 93% for two steps; and (I) H₂, 10% Pd/C, MeOH, 91%; and wherein steps xii-xviii correspond to steps xii-xviii as previously described herein for the preparation of the compound of Formula VI.

In an embodiment, the 2′,3′-dihydroxyls of D-ribose can be protected with an acetonide and the 5′-OH subsequently with a tert-butyldiphenylsilyl (TBDPS) group. A Wittig reaction of the product with CH₂=PPh₃ can give compound 17, whose 4′-OH can be oxidized with SO₃-pyridine, followed by an addition of a vinyl group to give diene 18. A ring closing metathesis reaction can produce cyclopentene compound 19 with a tertiary allylic —OH, which can be subjected to a pyridinium dichromate (PDC) mediated oxidation, thereby affording cyclopentenone 20. Cyclopentenone 20 can be stereoselectively reduced to compound 21 with a β-OH at the 1′-position. A Mitsunobu reaction of 21 with 6-chloropurine, followed by aminolysis of the obtained chloride 22 and 5′-deprotection of the silyl group, can produce the key cyclopentene intermediate 23, corresponding to the cyclopentane analog 13 in described herein for the preparation of the compound of Formula VI (e.g., Reaction Scheme I). Steps xii-xviii as previously described herein for the preparation of the compound of Formula VI can be used to transform compound 23 to the product characterized by Formula VII. In an embodiment, the yield of the compound of Formula VII from D-ribose can be greater than about 20 wt. %, alternatively greater than about 30 wt. %, or alternatively greater than about 40 wt. %, based on the theoretical amount of compound of Formula VII that could be obtained from D-ribose by following the indicated reaction scheme.

In an embodiment, the compound of Formula VII can be hydrogenated from the less hindered up side of the cyclopentene ring. In such embodiment, the yield of the hydrogenation of the compound of Formula VII to compound epi-6 can be greater than about 70 wt. %, alternatively greater than about 80 wt. %, or alternatively greater than about 90 wt. %, based on the theoretical amount of compound epi-6 that could be obtained by hydrogenating the compound of Formula VII.

In an embodiment, the compositions comprising a DOT1L Methyl Transferase inhibitor can be prepared via any suitable method or process. The components of the compositions comprising a DOT1L Methyl Transferase inhibitor (e.g., DOT1L Methyl Transferase inhibitor, carrier fluid, additives, etc.) can be combined using any mixing device compatible with the composition, e.g., that does not alter or destroy the components of the compositions, such as for example the DOT1L Methyl Transferase inhibitor, etc. In some embodiments, the carrier fluid can comprise water, dimethyl sulfoxide, and the like, or combinations thereof.

In an embodiment, the compositions comprising a DOT1L Methyl Transferase inhibitor can be used for the treatment of an individual having mixed lineage leukemia (MLL) and/or breast cancer (BC), wherein the composition comprising a DOT1L Methyl Transferase inhibitor can be a pharmaceutical composition.

In an embodiment, a method of treating an individual having MLL and/or BC can comprise administering to the individual an effective amount or a therapeutically effective amount of the composition comprising a DOT1L Methyl Transferase inhibitor, wherein the composition comprising a DOT1L Methyl Transferase inhibitor can be a pharmaceutical composition, thereby ameliorating, deterring and/or preventing MLL and/or BC in said individual. For purposes of the disclosure herein, an “effective amount” or “therapeutically effective amount” of composition comprising a DOT1L Methyl Transferase inhibitor can be defined as an amount of composition comprising a DOT1L Methyl Transferase inhibitor that produces a therapeutic response or desired effect (e.g., stopping progress of MLL and/or BC, reversing MLL and/or BC, etc.) in some fraction of individuals to which it is administered.

In an embodiment, the composition comprising a DOT1L Methyl Transferase inhibitor can be a pharmaceutical composition. For purposes of the disclosure herein, a pharmaceutical composition generally refers to any composition that may be used on or in a body to prevent, deter, diagnose, alleviate, treat, and/or cure a disease in humans or animals.

In an embodiment, a method of treating an individual having MLL and/or BC can comprise administering to the individual a therapeutically effective amount of the DOT1L Methyl Transferase inhibitor, wherein the DOT1L Methyl Transferase inhibitor can be a component of a pharmaceutical composition, thereby ameliorating, deterring and/or preventing MLL and/or BC in said individual. In some embodiments, the DOT1L Methyl Transferase inhibitor can be administered intravenously. However, other ways of administering DOT1L Methyl Transferase inhibitor could be possible, such as oral, intramuscular, inhalation, etc., or any other suitable way for administering a pharmaceutical composition.

In an embodiment, a method of treating MLL and/or BC in a subject can comprise administering to the subject a therapeutically effective amount of a compound of Formula I, a compound of Formula II, a compound of Formula III, and/or a compound of Formula IV. In an alternative embodiment, the DOT1L Methyl Transferase inhibitor can be administered as a prodrug, wherein said prodrug comprises replacing RCOOH and/or RCONH₂ in a compound of Formula I, a compound of Formula II, a compound of Formula III, and/or a compound of Formula IV with an analogous alkyl ester, an aryl ester, and/or a heteroaryl ester.

In an embodiment, a method of detecting MLL and/or BC in a subject can comprise adding a diagnostically effective amount of a compound of Formula I, a compound of Formula II, a compound of Formula III, and/or a compound of Formula IV, a pharmaceutically acceptable salt thereof, a prodrug thereof, or combinations thereof, to an in vitro biological sample.

In an alternative embodiment, the method of detecting MLL and/or BC in a subject can comprise administering to the subject a therapeutically effective amount of a compound of Formula I, a compound of Formula II, a compound of Formula III, and/or a compound of Formula IV, a pharmaceutically acceptable salt thereof, a prodrug thereof, or combinations thereof. In an embodiment, the subject is human.

In some embodiments, a compound of Formula I, a compound of Formula II, a compound of Formula III, and/or a compound of Formula IV, a pharmaceutically acceptable salt thereof, a prodrug thereof, or combinations thereof can specifically inhibit methylation of histone H3-lysine79 (H3K79) residues located in nucleosome core structure.

In other embodiments, a method of treating MLL and/or BC in a subject can comprise administering to the subject a compound that is a structural mimic of a reaction intermediate of a compound of Formula I, a compound of Formula II, a compound of Formula III, and/or a compound of Formula IV, a pharmaceutically acceptable salt thereof, a prodrug thereof, or combinations thereof.

In an embodiment, a method of treating MLL and/or BC in a subject can comprise administering to the subject a therapeutically effective amount of a compound of Formula I, a pharmaceutically acceptable salt thereof, a prodrug thereof, or combinations thereof:

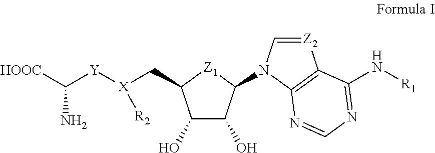

wherein R₁ can be H, methyl, or benzyl; R₂ can be 2-cyanoethyl, 2-methoxycarbonylethyl, or 2-iodoethyl; X can be N or S; wherein if X=S, R₂=O; Y can be C₃ or C₄, Z₁ can be O, S, N, or CH₂, and Z₂ can be N, or CR₄, wherein R₄ is a halogen, alkyl, aryl or a 5- or 6-membered heterocycle; and wherein said compound can be selective for DOT1L Methyl Transferase. In an embodiment, the compound of Formula I can be used in the manufacture of a medicament for the treatment of MLL and/or BC.

In an embodiment, a method of treating MLL and/or BC in a subject can comprise administering to the subject a therapeutically effective amount of a compound of Formula II, a pharmaceutically acceptable salt thereof, a prodrug thereof, or combinations thereof:

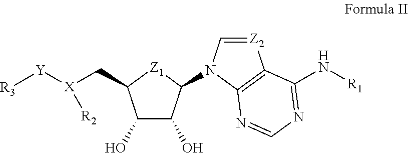

wherein R₁ can be H, alkyl, or benzyl; R₂ can be H, 2-cyanoethyl, 2-methoxycarbonylethyl, methyl, 2-iodoethyl, ethanol, butyl, or benzyl carbamate; X can be N, C, or S; wherein if X=S, R₂=O; and wherein if X=C, R₂ can also be equal to R₃ or R₁, and Y can also be equal to R₁, R₂ or R₃; Y can be C₁, C₂, C₃ or C₄; Z₁ can be 0, S, N, or CH₂; Z₂ can be N, or CR₄, wherein R₄ can be a halogen, alkyl, aryl or a 5- or 6-membered heterocycle; R₃ can be H or selected from the following:

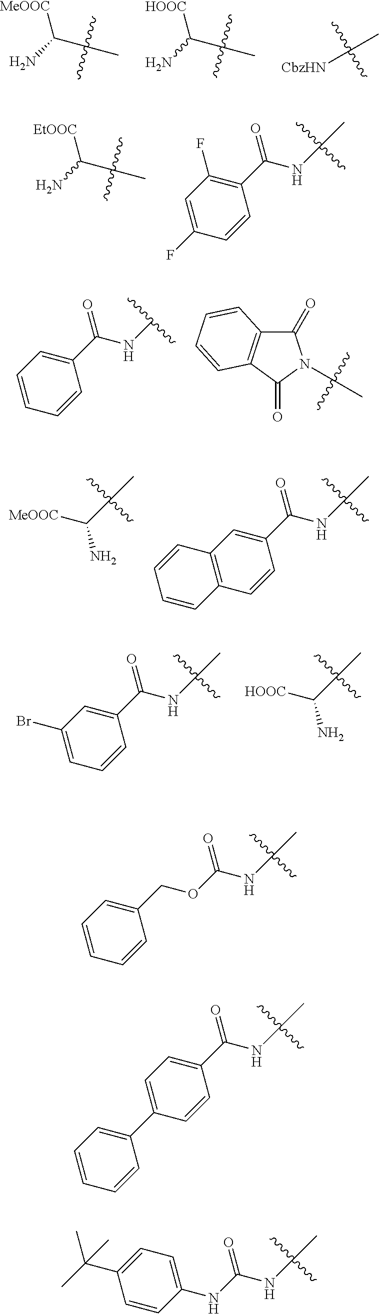

and wherein said compound can be selective for DOT1L Methyl Transferase. In an embodiment, the compound of Formula II can be used in the manufacture of a medicament for the treatment of MLL and/or BC.

In an embodiment, a method of treating MLL and/or BC in a subject can comprise administering to the subject a therapeutically effective amount of a compound of Formula III, a pharmaceutically acceptable salt thereof, a prodrug thereof, or combinations thereof:

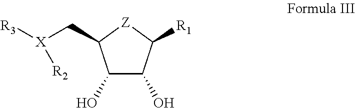

wherein R₁ can be H, or a substituted or nonsubstituted: alkyl, cycloalkyl, morpholino, aryl, biaryl, fused biaryl, benzyl; heterocycle, purine, pyrimidine, alcohol, amine, amide, aldehyde, ketone, thiol; ester, ethers, carboxylate, acyl halide, imide, amidine, nitrile, cyano, thioaldehyde, ketone, thione, thioester, thioether, hydrazines, or disulphide; Z can be CH₂; X can be C, N, O or S; wherein if X=O, R₂=O; R₃ can be H, O, or R₁; R₂ can be H, O, or R₁; or R₃ and R₂ can be cyclized together to form a substituted or nonsubstituted: alkyl, cycloalkyl, aryl, biaryl, fused biaryl, benzyl; heterocycle, purine, pyrimidine; and wherein said substituent may be selected from R₁, R₂, R₃, X, halide; or combinations thereof; and wherein said compound can be selective for DOT1L Methyl Transferase. In an embodiment, the compound of Formula III can be used in the manufacture of a medicament for the treatment of MLL and/or BC.

In an embodiment, a method of treating MLL and/or BC in a subject can comprise administering to the subject a therapeutically effective amount of a compound of Formula IV, a pharmaceutically acceptable salt thereof, a prodrug thereof, or combinations thereof:

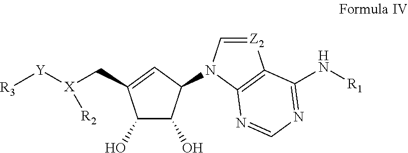

wherein R₁ can be H, alkyl, or benzyl; R₂ can be H, 2-cyanoethyl, 2-methoxycarbonylethyl, methyl, 2-iodoethyl, ethanol, butyl, or benzyl carbamate; X can be N, C, or S; wherein if X=S, R₂=O; and wherein if X=C, R₂ can also be equal to R₃ or R₁, and Y can also be equal to R₁, R₂ or R₃; Y can be C₁, C₂, C₃ or C₄; Z₂ can be N, or CR₄, wherein R₄ can be a halogen, alkyl, aryl or a 5- or 6-membered heterocycle; R₃ can be H or selected from the following:

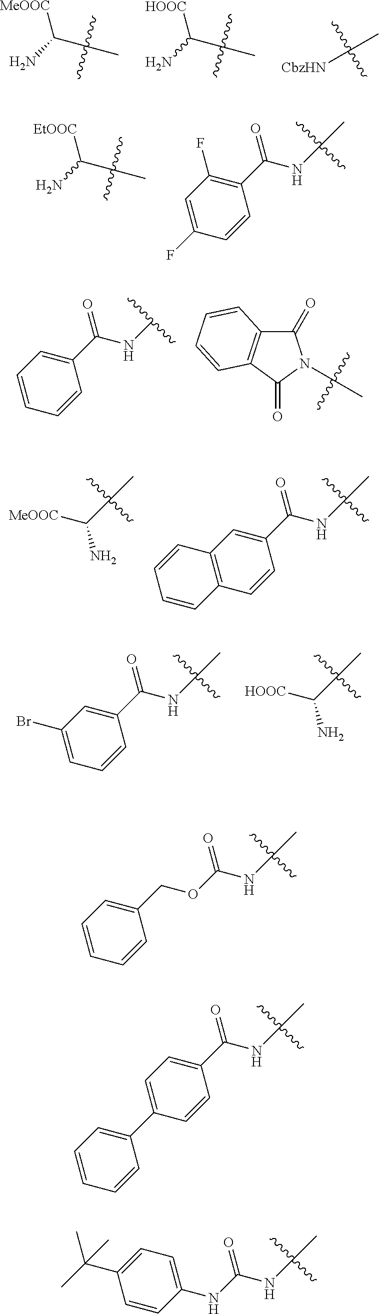

and wherein said compound can be selective for DOT1L Methyl Transferase. In an embodiment, the compound of Formula IV can be used in the manufacture of a medicament for the treatment of MLL and/or BC.

In an embodiment, a method of treating MLL and/or BC in a subject can comprise administering to the subject a therapeutically effective amount of a compound of Formula VI, a pharmaceutically acceptable salt thereof, a prodrug thereof, or combinations thereof:

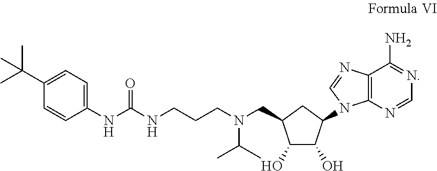 [SMILES: CC(C)N(CCCNC(=O)Nc1ccc(C(C)(C)C)cc1)C[C@H]1C[C@@H](n2cnc3c(N)ncnc32)[C@H](O)[C@@H]1O]

In an embodiment, the compound of Formula VI can be used in the manufacture of a medicament for the treatment of MLL and/or BC.

In an embodiment, a method of treating MLL and/or BC in a subject can comprise administering to the subject a therapeutically effective amount of a compound of Formula VII, a pharmaceutically acceptable salt thereof, a prodrug thereof, or combinations thereof:

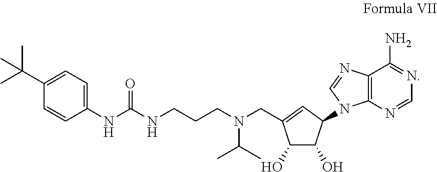 [SMILES: CC(C)N(CCCNC(=O)Nc1ccc(C(C)(C)C)cc1)CC1=C[C@@H](n2cnc3c(N)ncnc32)[C@H](O)[C@@H]1O]

In an embodiment, the compound of Formula VII can be used in the manufacture of a medicament for the treatment of MLL and/or BC.

In another embodiment, a method of targeting breast cancer cells can comprise treating breast cancer cells with a compound of Formula I, a compound of Formula II, a compound of Formula III, and/or a compound of Formula IV, a pharmaceutically acceptable salt thereof, a prodrug thereof, or combinations thereof; wherein the breast cancer cells comprise an elevated amount of DOTL1 compared to non-cancerous breast cells. In such embodiment, the breast cancer cells can be in vivo or in vitro. In such embodiment, the breast cancer cells can be breast cancer stem cells. In some embodiments, the breast cancer cells undergoing treatment with a compound of Formula I, a compound of Formula II, a compound of Formula III, and/or a compound of Formula IV, a pharmaceutically acceptable salt thereof, a prodrug thereof, or combinations thereof can be MDA-MB231 cells; BT549 cells; and/or MCF-7 cells.

In another embodiment, a method of targeting leukemia cells can comprise treating leukemia cells with a compound of Formula I, a compound of Formula II, a compound of Formula III, and/or a compound of Formula IV, a pharmaceutically acceptable salt thereof, a prodrug thereof, or combinations thereof; wherein the leukemia cells comprise an elevated amount of DOTL1 compared to non-cancerous cells. In such embodiment, the leukemia cells can be in vivo or in vitro. In some embodiments, the leukemia cells undergoing treatment with a compound of Formula I, a compound of Formula II, a compound of Formula III, and/or a compound of Formula IV, a pharmaceutically acceptable salt thereof, a prodrug thereof, or combinations thereof can be MV4-11 cells; Molm-13 cells; RS4-11 cells; SEM cells; KOPN-8 cells; and/or THP-1 cells.

In an embodiment, a method of targeting breast cancer cells can comprise inhibiting the methylation of histone H3-lysine79 (H3K79) residues located in nucleosome core structure, by administering a compound of Formula I, a compound of Formula II, a compound of Formula III, and/or a compound of Formula IV, a pharmaceutically acceptable salt thereof, a prodrug thereof, or combinations thereof. In an embodiment, a method of targeting breast cancer cells can further comprise at least one of: inhibiting cell self-renewable ability and inducing differentiation of breast cancer stem cells; reversing disregulated gene expression of claudins, E-cadherin, and/or epithelial mesenchymal transition traits; and the like.

In an embodiment, a method of targeting MLL-leukemia cancer cells can further comprise at least one of: inhibiting cell self-renewable ability and inducing differentiation of leukemia cancer stem cells; reversing disregulated gene expression of HoxA9 and Meis1; and the like.

In an embodiment, a method of detecting BC can comprise adding to an in vitro biological sample a diagnostically effective amount of a compound of Formula I, a compound of Formula II, a compound of Formula III, and/or a compound of Formula IV, a pharmaceutically acceptable salt thereof, a prodrug thereof, or combinations thereof.

In another embodiment, a method of detecting BC in a subject can comprise administering to the subject a therapeutically effective amount of a compound of Formula I, a compound of Formula II, a compound of Formula III, and/or a compound of Formula IV, a pharmaceutically acceptable salt thereof, a prodrug thereof, or combinations thereof.

In an embodiment, a method of detecting MLL can comprise adding to an in vitro biological sample a diagnostically effective amount of a compound of Formula I, a compound of Formula II, a compound of Formula III, and/or a compound of Formula IV, a pharmaceutically acceptable salt thereof, a prodrug thereof, or combinations thereof.

In another embodiment, a method of detecting MLL in a subject can comprise administering to the subject a therapeutically effective amount of a compound of Formula I, a compound of Formula II, a compound of Formula III, and/or a compound of Formula IV, a pharmaceutically acceptable salt thereof, a prodrug thereof, or combinations thereof.

In an embodiment, the method of treating an individual having MLL and/or BC with a composition comprising a DOT1L Methyl Transferase inhibitor as disclosed herein advantageously displays improvements in one or more outcomes when compared to a treatment method with an otherwise similar composition lacking the DOT1L Methyl Transferase inhibitor. Currently there have been no effective chemotherapeutics to treat relapsed (or metastatic) MLL or BC.

In an embodiment, an individual undergoing treatment for MLL and/or BC with a composition comprising a DOT1L Methyl Transferase inhibitor as disclosed herein may experience less side effects or toxicity than an individual undergoing different chemotherapy treatment for MLL and/or BC. Additional advantages of the compositions comprising a DOT1L Methyl Transferase inhibitors and treatment methods of using same can be apparent to one of skill in the art viewing this disclosure.

### EXAMPLES

The embodiments having been generally described, the following examples are given as particular embodiments of the disclosure and to demonstrate the practice and advantages thereof. It is understood that the examples are given by way of illustration and are not intended to limit the specification or the claims in any manner.

All reagents were purchased from Alfa Aesar (Ward Hill, Mass.) or Aldrich (Milwaukee, Wis.). All compounds were characterized by ¹H spectrum on a Varian (Palo Alto, Calif.) 400-MR spectrometer. The purities were determined by a Shimadzu Prominence HPLC using a Zorbax C18 column (4.6×250 mm; methanol:water=70:30; flow rate=1 mL/min; monitored at 254 and 280 nm). The purities of compound of Formula VI, compound of Formula VII, and compound epi-6 were found to be >95%. Identities of compound of Formula VI, compound of Formula VII, and compound epi-6 were confirmed with high resolution mass spectra (HRMS) using a ThermoFisher LTQ-Orbitrap mass spectrometer. Control compounds 4 and SAH of FIG. 1 were obtained and characterized as described in J. Am. Chem. Soc. 2011, 133, pp. 16746-16749 and J. Med. Chem. 2012, 55, pp. 8066-8074, each of which is incorporated by reference herein in its entirety.

### Example 1

### Biological Activity Evaluation

Compound of Formula VI, compound of Formula VII, and compound epi-6 were tested for their inhibitory activities against recombinant human DOT1L, together with compound 4 of FIG. 1 as a control, and the data are displayed in Table 1. Expression, purification and inhibition of recombinant human DOT1L (catalytic domain (AA) 1-472) were performed according to previously published methods (J. Am. Chem. Soc. 2011, 133, pp. 16746-16749 and J. Med. Chem. 2012, 55, pp. 8066-8074). In brief, compounds with concentrations ranging from 1 nM to 100 μM were incubated with DOT1L (100 nM), 1.5 μM oligo-nucleosome in 20 μL of 20 mM Tris buffer (containing 1 mM EDTA, 0.5 mM DTT and 50 μg/mL BSA, pH=8.0) for 10 min. 0.76 μM (=Kₘ) of ³H-SAM (10 Ci/mM; Perkin-Elmer) was added to initiate the reaction. After 30 min at 30° C., the reaction was stopped by adding SAH (100 μM). 15 μL of reaction mixture was transferred to P81 filter paper (Whatman) that binds histone H3 protein, washed 3× with 50 mM NaHCO₃, dried, and placed into a scintillation vial containing scintillation cocktail (2 mL), which was measured with a Beckman LS-6500 scintillation counter. Kᵢ values were calculated using the Morrison tight binding model fitting or standard sigmoidal dose response curve fitting (for less potent inhibitors) in Prism 5.0. Enzyme inhibition assays for PRMT1, CARM1 and SUV39H1 were performed.

TABLE 1

| | DOT1L | PRMT1 | CARM1 | SUV39H1 |
|---|---|---|---|---|
| SAH | 0.16 | 0.40 | 0.86 | 4.9 |
| Compound 4 | 0.00072 | >50 | >50 | >50 |
| (of FIG. 1) | | | | |
| compound of Formula VI | 0.0011 | >50 | >50 | >50 |
| compound of Formula VII | 0.0013 | >50 | >50 | >50 |
| compound epi-6 | >50 | >50 | >50 | >50 |

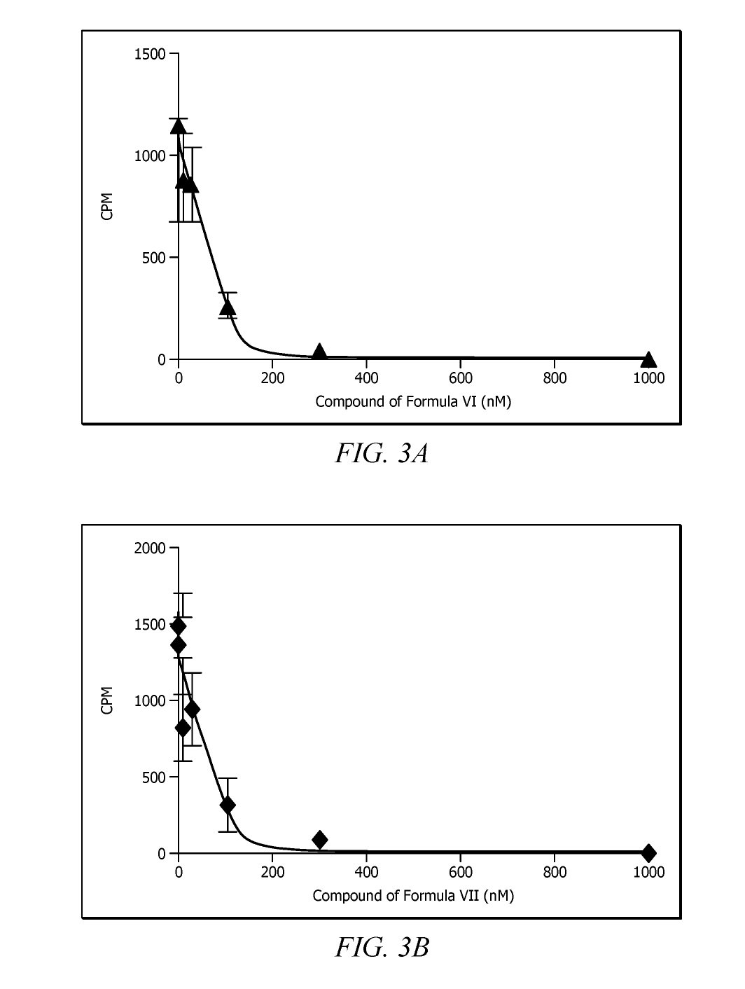

Table 1 displays Kᵢ [μM] values against DOT1L and other HMTs (e.g., PRMT1, CARM1, and SUV39H1). As shown in FIG. 3A and FIG. 3B, carbocyclic analogs (e.g., compound of Formula VI and compound of Formula VII, respectively) exhibited a very high potency against the enzyme with Kᵢ values of 1.1 and 1.3 nM, respectively, showing a comparable activity as inhibitor 4 (Kᵢ=0.72 nM). In addition to the compound of Formula VI (with a similar structure as compound 4), the flexible linker between the 5′-position and the urea group in the compound of Formula VII may allow both its adeninylcyclopentene and N-tert-butylphenyl urea moieties occupy the optimal binding sites in DOT1L. It was found that there is little binding affinity difference among compound 4, compound of Formula VI and compound of Formula VII. However, compound epi-6 was found to be inactive against DOT1L with a Kᵢ value of >50 μM, showing the corresponding two groups of compound epi-6 cannot be in the favorable positions due to the trans-orientated 5′-sidechain (relative to 1′-adeninyl).

Next, HMT enzyme selectivity of compound of Formula VI and compound of Formula VII was evaluated. As with their adenosine-containing analog 4, these two potent DOT1L inhibitors (i.e., compound of Formula VI and compound of Formula VII) were found to have no activity against three representative histone methyltransferases CARM1, PRMT1 and SUV39H1, as shown in Table 1. This is in contrast to another DOT1L inhibitor SAH, which has broad activity against all these HMTs. The improved selectivity of compound of Formula VI and compound of Formula VII may also be due to the hydrophobic urea-containing sidechain. Previous crystallographic studies show the SAM amino acid binding pocket of DOT1L undergoes a large conformational change, the urea functionality of these inhibitors forms two hydrogen bonds with Asp161 and the 4-tert-butylphenyl group is favorably located in a newly formed hydrophobic pocket of DOT1L, showing the structural basis for the high selectivity over other histone methyltransferases.

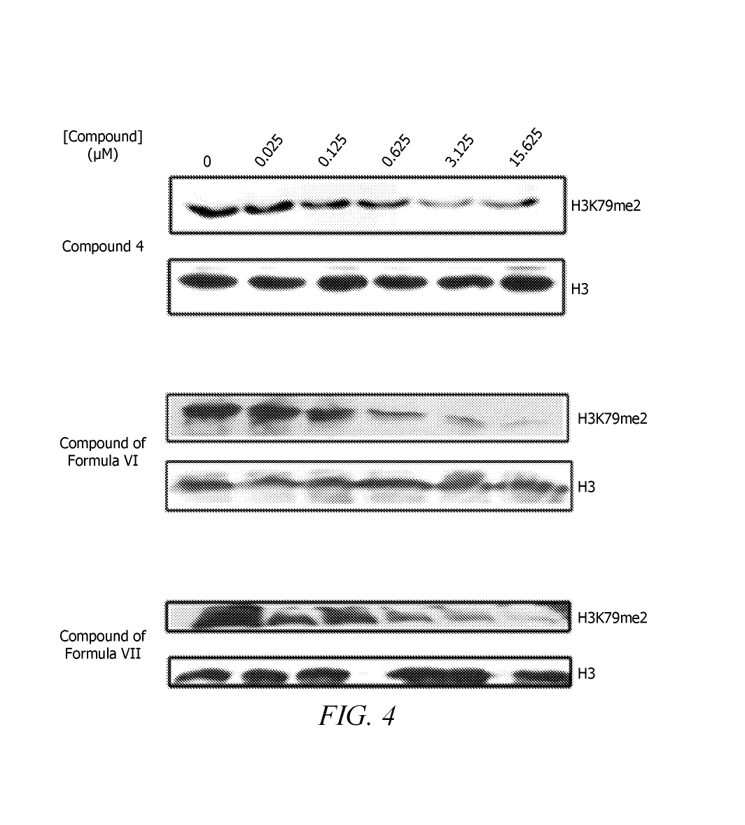

Further, FIG. 4 shows that compound of Formula VI and compound of Formula VII are able to block H3K79 methylation in human leukemia cell line MV4-11 in a dose-dependent manner. Inhibitor 4 (e.g., compound 4 of FIG. 1) was also used in the study. MV4-11 cells in the exponential growth phase were incubated in the presence of increasing concentrations of compounds (0.025-15.625 μM). Cells (2×10⁶) were harvested at day 4 and histones were extracted with the EpiQuik™ total histone extraction kit (Epigentek) according to the manufacturer's protocol. Equal amounts of histones (2 μg) were separated with SDS-PAGE and transferred to a piece of PVDF membrane. The Western Blots were incubated with primary antibodies against dimethylated H3K79 and Histone H3 (Cell Signaling), followed by secondary antibody (anti-rabbit IgG) coupled with horseradish peroxidase (HRP), and detected with Supersignal West Dura substrate (Thermo Scientific).

The IC₅₀ values of compound of Formula VI and compound of Formula VII were estimated to be ˜0.2 μM, showing a similar cell activity as compound 4. Thus, the IC₅₀ values of these competitive inhibitors are much higher than their corresponding enzyme Kᵢ values (˜1 nM), which is mainly due to a considerably higher concentration of SAM in cells (˜300 μM) as compared to that used in the enzyme inhibition assay (0.76 μM=the Kₘ value). In addition, cell membrane permeability as well as other factors including stability may also affect cell activities of these compounds.

### Example 2

### Metabolic Stability Evaluation

Compound of Formula VI and compound of Formula VII were investigated for their metabolic stability. Ribose-containing inhibitors are metabolically unstable, and the compound of Formula VI and the compound of Formula VII address the metabolic stability by providing non-ribose containing compounds, i.e., removing the reactive/labile ribose group.

For microsome stability assay, pooled human liver microsomes were purchased from Invitrogen. To a 100 mM phosphate buffer solution (450 μL) containing microsomes (the final concentration of 0.5 mg/mL) and MgCl₂ (5 mM) was add 5 μL of 200 μM a test compound or verapamil (as the control compound) at 37° C. 50 μL of NADPH (1 mM final concentration) solution in the same buffer was added to initiate the reaction. Aliquots of 50 μL were taken from the reaction solution at 0, 15, 30, 45 and 60 min. The reaction was stopped by the addition of 3 volumes of methanol. Samples were centrifuged at 16,000 g for 10 minutes to precipitate protein. Aliquot of 100 μL of the supernatant was used for LC/MS/MS analysis to determine the remaining amount of the test compound, using Shimadzu HPLC [Phenomenex 5μ C18 (2.0×50 mm) column; Mobile phase: 0.1% formic acid in acetontrile (A) and 0.1% formic acid in water (B)] followed by AB Sciex API4000 mass spectrometer [parameters: ion source, Turbo spray; ionization model, ESI; scan type, MRM; collision gas, 6 L/min; curtain gas, 30 L/min; nebulize gas, 50 L/min; auxiliary gas, 50 L/min; temperature, 500° C.; ionspray voltage, +5500 v (positive MRM)]. All experiments were performed in duplicate.

For human plasma stability assay, to human plasma (500 μL) was added a test compound to a final concentration of 5 μM and incubated at 37° C. at approximately 60 rpm on an orbital shaker. Aliquots of 50 μL were taken from the reaction solution at 0, 15, 30, 45 and 60 minutes. The reaction was stopped by the addition of 6 volumes of cold acetonitrile. Samples were centrifuged at 20,000 g for 15 minutes to precipitate protein. An aliquot of 150 μL of the supernatant was used to determine the remaining amount of the test compound, using the same LC/MS/MS method described above. All experiments were performed in duplicate.

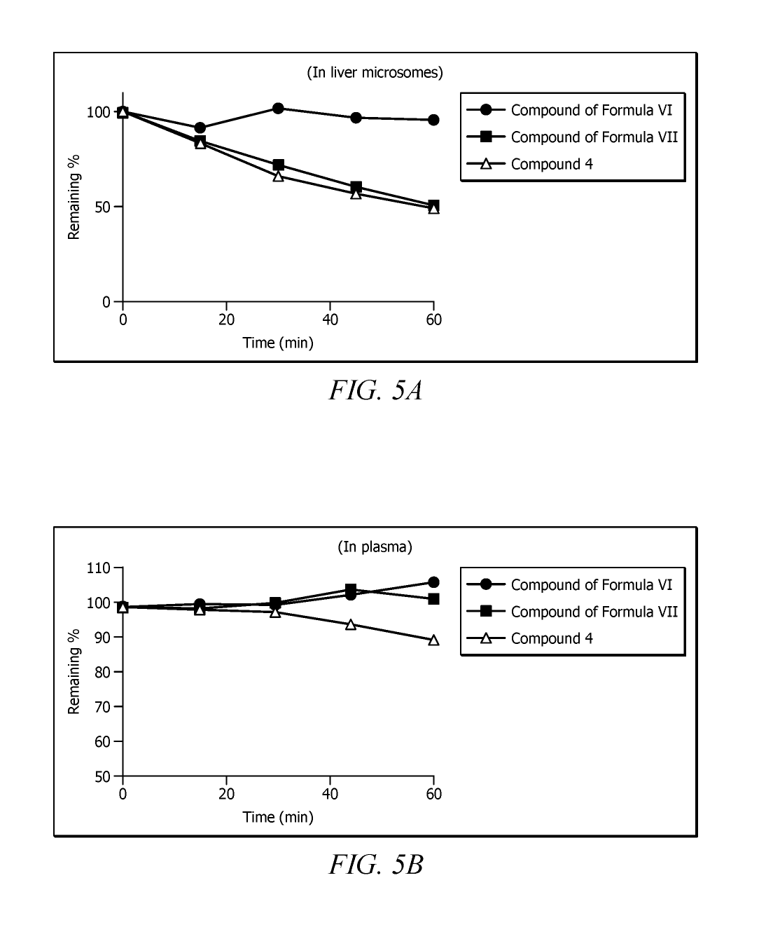

An in vitro metabolic stability of compound of Formula VI and compound of Formula VII, which are potent DOT1L inhibitors, in human plasma and liver microsomes was performed, the latter of which are mainly responsible for drug metabolism. These two assays are standard indicators for predicting in vivo pharmacokinetic parameters of a compound. Compound 4 of FIG. 1 was included in the study as a comparison. As shown in FIG. 5A and FIG. 5B, although the ribose-containing compound 4 is reasonably stable in human plasma with ˜90% remaining after 1 h, it is quickly degraded in the presence of human liver microsomes with only ˜50% unchanged after 1 h. The intrinsic clearance (CLint) of compound 4 is 24.0 μL/min/mg protein (microsomes). This is in line with a previous study for compound 1 of FIG. 1, showing a quick degradation and a short half-life in vivo. The cyclopentane-containing compound of Formula VI exhibits a very high metabolic stability in both plasma and liver microsomes, with a CLint value of only 0.36 μL/min/mg protein. Unlike compound of Formula VI, the cyclopentene compound of Formula VII can also be metabolized by microsomes with about half the amount remaining after 1 h treatment (CLint=22.5 μL/min/mg protein), although it is stable in human plasma containing few metabolic enzymes (FIG. 5A and FIG. 5B). This may be due to the C═C double bond in compound of Formula VII that may be oxidized by, for example, cytochrome P450 in microsomes. These results show changing the metabolically labile ribose ring to the cyclopentane group is an effective strategy to produce better drug candidates with favorable pharmacokinetic properties.

Thus, cyclopentane-containing compound of Formula VI, an analog of a potent DOT1L inhibitor 4, was synthesized efficiently with an overall yield of 19.3%, starting from readily available D-ribose. The compound of Formula VI potently inhibits human DOT1L with a Ki value of 1.1 nM, and is inactive against other HMTs. In addition, it possesses potent activity in inhibiting cellular H3K79 methylation with an IC50 of ˜200 nM. Further, compound of Formula VI is metabolically stabile, and does not undergo degradation by human plasma and liver microsomes, thereby this class of compounds may be further developed for targeting MLL leukemia.

### Example 3

The compounds of Reaction Scheme I were synthesized as follows.

Compound 8.

To a solution of D-ribose (7.5 g, 50 mmol) in cyclohexanone (50 mL) was added concentrated H₂SO₄ (0.5 mL). The reaction mixture was stirred for 6 h at room temperature, after which the solvent was removed under reduced pressure. The residue was dissolved in EtOAc (100 mL), washed with saturated NaHCO₃ (30 mL) and brine (30 mL). The organic layer was dried over Na₂SO₄ and concentrated to give a light brown oil, which was dissolved in 100 mL tetrahydrofuran (THF). Vinylmagnisum bromide (250 mL, 1 M in THF) was added dropwise at −78° C. The reaction mixture was allowed to warm to room temperature and stirred for 12 h. The reaction was quenched by adding saturated NH₄CI (40 mL) and the aqueous layer was extracted with EtOAc (3×40 mL). The combined organic phases were washed with brine (20 mL), dried over Na₂SO₄, and concentrated under reduced pressure. The residue was purified with column chromatography (silica gel, EtOAc/Hexanes 2:1) to give compound 8 as a colorless oil (8.65 g, 70%). ¹H NMR (400 MHz, CDCl₃): δ 6.16-6.03 (m, 1H), 5.40 (d, J=17.6 Hz, 1H), 5.30 (d, J=10.8 Hz, 1H), 4.39-4.35 (m, 1H), 4.14-4.12 (m, 1H), 4.07-4.04 (m, 1H), 3.97-3.91 (m, 2H), 3.77-3.72 (m, 1H), 1.66-1.39 (m, 10H).

Compound 9.

To a solution of compound 8 (2.32 g, 9 mmol) in MeOH/H₂O (80 mL, 5:1) was added NalO₄ (3.86 g, 18 mmol) slowly at 0° C. The reaction was warmed to room temperature and stirred for 1 h. Upon filtering off the solid, MeOH was removed under reduced pressure. EtOAc (80 mL) was added, washed with brine (20 mL), dried over Na₂SO₄, and concentrated to give an aldehyde, which was used for next step without further purification. To a suspension of Ph₃PCH₃Br (8.0 g, 22.5 mmol) in THF (45 mL) was added t-BuOK (2.52 g, 22.5 mmol) slowly at 0° C. After stirred for 1 h at room temperature, the reaction mixture was cooled to 0° C. and a solution of the crude aldehyde in THF (9 mL) was added. The reaction mixture was stirred overnight and quenched by adding water (25 mL). The aqueous layer was extracted with EtOAc (3×20 mL) and the combined organic phases were washed with brine (20 mL), dried over Na₂SO₄, concentrated, and purified with column chromatography (silica gel, EtOAc/Hexanes 1:6) to give compound 9 as a colorless oil (1.77 g, 87%). ¹H NMR (400 MHz, CDCl₃): δ 6.14-5.98 (m, 2H), 5.46-5.22 (m, 4H), 4.64 (m, 1H), 4.18 (m, 1H), 4.01 (m, 1H), 1.79 (d, J=4.4 Hz, 1H), 1.67-1.37 (m, 10H).

Compound 10.

A solution of compound 9 (1.1 g, 4.91 mmol) in CH₂Cl₂ (100 mL) was degassed, followed by addition of 2^{nd} generation Grubbs' catalyst (172 mg, 5 mmol %). The reaction mixture was stirred overnight at room temperature and concentrated under reduced pressure to give a brown dark residue, which was dissolved in CH₂Cl₂ (20 mL). Dess-Martin periodinane (DMP, 3.11 g, 7.34 mmol) and NaHCO₃ (619 mg, 7.34 mmol) were added into the solution. After 2 h, the reaction was quenched by adding saturated Na₂S₂O₃ (30 mL) and ether (100 mL) and stirred for 30 min. The separate organic layer was dried over Na₂SO₄, concentrated and the residue purified with column chromatography (silica gel, EtOAc/Hexanes 1:8) to give compound 10 as a colorless oil (821 mg, 86%). ¹H NMR (400 MHz, CDCl₃): δ 7.60 (dd, J=2.4, 3.6 Hz, 1H), 6.20 (d, J=5.2 Hz, 1H), 5.27-5.24 (m, 1H), 4.45 (d, J=5.2 Hz, 1H), 1.67-1.37 (m, 10H).

Compound 11.

To a suspension of CuBr-Me₂S complex (57 mg) in THF (15 mL) was added vinylmagnesium bromide (4.06 mL, 4.1 mmol, 1 M solution in THF) slowly at −78° C. After 15 min, a solution of compound 10 (630 mg, 3.25 mmol), chlorotrimethylsilane (845 μL), and hexamethylphosphoramide (1.45 mL) in THF (5 mL) was added slowly. The reaction was further stirred at −78° C. for 5 h, allowed to warm to 0° C. and quenched with saturated NH₄CI (10 mL). The aqueous layer was extracted with EtOAc (3×10 mL) and the combined organic phases were washed with brine (10 mL), dried over Na₂SO₄, concentrated under reduced pressure, and purified with column chromatography (silica gel, EtOAc/Hexanes 1:10) to give the 1,4-addition product as a white oil [641 mg, 89%, ¹H NMR (400 MHz, CDCl₃): δ 5.81 (m, 1H), 5.14-5.06 (m, 2H), 4.60 (d, J=4.8 Hz, 1H), 4.17 (d, J=5.2 Hz, 1H), 3.10 (t, J=4.8 Hz, 1H), 2.82 (dd, J=19.2, 8.8 Hz, 1H), 2.31-2.22 (m, 1H), 1.65-1.37 (m, 10H)].

To a solution of the product thus obtained (666 mg, 3.0 mmol) in MeOH (40 mL) was added CeCl₃.7H₂O (780 mg, 2.1 mmol) and NaBH₄ (224.2 mg, 6 mmol) at −15° C. The reaction mixture was warmed to room temperature and stirred for 1 h. Upon removal of the solvent, water was added to the residue and pH adjusted to 5 with acetic acid. The product was extracted with EtOAc (3×15 mL). The combined organic phases were washed with brine (10 mL), dried over Na₂SO₄, concentrated, and purified with column chromatography (silica gel, EtOAc/Hexanes 1:4) to give compound 11 as a colorless oil (671 mg, 99%). ¹H NMR (400 MHz, CDCl₃): δ 5.71 (m, 1H), 5.19-5.02 (m, 2H), 4.48-4.46 (m, 1H), 4.07-4.02 (m, 1H), 2.76-2.70 (m, 1H), 2.48 (d, J=7.2 Hz, 1H), 1.90-1.86 (m, 2H), 1.69-1.35 (m, 10H).

Compound 12.

To a solution of compound 11 (300 mg, 1.34 mmol), 6,6-di-Boc-protected adenine (898 mg, 2.7 mmol) and Ph₃P (807 mg, 3.1 mmol) in THF (20 mL) was added diisopropyl azodicarboxylate (DIAD, 625 μL) dropwise at 0° C. The reaction mixture was allowed to warm to room temperature and stirred for 3 days. Upon removal of the solvent, the residue was subjected to a flash column chromatography (silica gel, EtOAc/Hexanes 1:3) to give compound 12 as a white foam (530 mg, 73%). ¹H NMR (400 MHz, CDCl₃): δ 8.82 (s, 1H), 8.09 (s, 1H), 5.94 (m, 1H), 5.21-5.14 (m, 2H), 5.12-5.06 (m, 1H), 4.83-4.79 (m, 1H), 4.64-4.62 (m, 1H), 2.83-2.79 (m, 1H), 2.57-2.47 (m, 1H), 1.91-1.87 (m, 2H), 1.67-1.37 (m, 10H), 1.44 (s, 18H).

Compound 13.

A solution of compound 12 (1.08 g, 2 mmol) in CH₂Cl₂ (10 mL) was cooled to −78° C., into which O₃ was bubbled until the solution became light blue. Upon removal of the excess O₃ by passing N₂, Me₂S (5 mL) was added into the solution. The reaction mixture was warmed slowly to room temperature over 1 h and concentrated to give a syrup, which was dissolved in MeOH (8 mL). NaBH₄ (148 mg, 4 mmol) was added into the solution at 0° C. and stirred for 1 h before adding water. The product was extracted with EtOAc (3×10 mL) and the combined organic phases were washed with brine (10 mL), dried over Na₂SO₄, filtered and concentrated under reduced pressure to give a colorless oil. It was dissolved in CH₂Cl₂ (4.5 mL) at 0° C., and trifluoroacetic acid (TFA, 0.5 mL) was added dropwise. After stirring for 2 h at room temperature, the solvent was removed under reduced pressure and CH₂Cl₂ (10 mL) was added. This step was repeated for 3 times and the resulting residue was purified with column chromatography (silica gel, 5% methanol in ethyl acetate) to give compound 13 as a white solid (635 mg, 99%). ¹H NMR (400 MHz, CDCl₃): δ 8.40 (s, 1H), 7.88 (s, 1H), 5.01 (t, J=6.4 Hz, 1H), 4.81-4.79 (m, 1H), 4.69-4.68 (m, 1H), 4.44 (br, 1H), 3.82-3.79 (m, 2H), 2.42 (m, 1H), 1.80-1.78 (m, 2H), 1.68-1.37 (m, 10H).

Compound 14.

To a solution of compound 13 (553 mg, 1.6 mmol), phthalimide (470 mg, 3.2 mmol) and Ph₃P (837 mg, 3.2 mmol) in THF (15 mL) was added diisopropyl azodicarboxylate (646 mg, 3.2 mmol) slowly at 0° C. After stirring overnight at room temperature, the solvent was evaporated and the resulting residue was purified with column chromatography (silica gel, EtOAc) to give the product (698 mg, 92%) as a white foam, which was refluxed with hydrazine monohydrate (480 mg, 9.6 mmol) in EtOH (15 mL) for 2 h. Upon cooling and filtering off an insoluble material, the filtrate was concentrated under reduced pressure and purified with column chromatography (silica gel, EtOAc:methanol (9:1) containing 1% triethylamine) to give a primary amine as a syrup (500 mg, 99%). To a solution of the product (690 mg, 2.0 mmol) in MeOH (20 mL) were added HOAc (1.16 mL), acetone (2.0 mL) and NaBCNH₃ (730 mg, 10.0 mmol) at 0° C. After stirring overnight, the solvent was removed under reduced pressure. Water (10 mL) was added and the product was extracted with EtOAc (6×20 mL). The combined organic phases were dried over Na₂SO₄, concentrated under reduced pressure, and purified with column chromatography (silica gel, EtOAc:methanol (9:1) containing 1% triethylamine) to give compound 14 as a white oil (733 mg, 95%). ¹H NMR (400 MHz, CDCl₃): δ 8.42 (s, 1H), 7.88 (s, 1H), 4.94-4.93 (m, 1H), 4.58-4.57 (m, 1H), 4.46-4.45 (m, 1H), 4.06 (m, 1H), 2.84-2.82 (m, 2H), 2.41-2.39 (m, 2H), 2.12-2.10 (m, 1H), 1.66-1.35 (m, 10H), 1.09 (d, J=6.0 Hz, 6H).

Compound 15.

Compound 14 (1.55 g, 4.0 mmol) was refluxed with methyl acrylate (2 mL) in MeOH (12 mL) for 3 days to produce a 1,4-adduct in almost quantitative yield. The crude product was reduced with LiAlH₄ (152 mg, 4.0 mmol) in THF (25 mL) at −15° C. for 1 h and room temperature for 3 h. The reaction mixture was quenched by adding of EtOAc at 0° C. and further stirred for 2 h. The precipitation was filtered off and thoroughly washed with EtOAc. The combined organic phases were evaporated under reduced pressure and purified with column chromatography (silica gel, EtOAc:methanol 9:1) to give compound 15 as a white oil (1.6 g, 91%). ¹H NMR (400 MHz, CDCl₃): δ 8.40 (s, 1H), 7.82 (s, 1H), 4.96-4.93 (m, 1H), 4.57-4.56 (m, 1H), 4.45-4.43 (m, 1H), 4.07 (m, 1H), 3.76-3.74 (m, 2H), 3.19-3.16 (m, 1H), 2.72-2.46 (m, 4H), 2.05-1.99 (m, 1H), 1.82-1.37 (m, 14H), 1.04 (d, J=6.4 Hz, 3H), 1.03 (d, J=6.4 Hz, 3H).

### 1-(3-((((1R,2R,3S,4R)-4-adenosyl-2,3-dihydroxycyclopentyl)methyl)(isopropyl)-amino)propyl)-3-(4-(tert-butyl)phenyl)urea (compound of Formula VI)

Using steps xii and xiii (Reaction Scheme I) described above, compound 15 (700 mg, 1.6 mmol) was subjected to a Mitsunobu reaction with phthalimide, followed by treatment with hydrazine, to give compound 16 as a white foam (640 mg, 91%). To a solution of compound 16 (222 mg, 0.5 mmol) in CH₂Cl₂ (4 mL), triethylamine (77 μL, 0.55 mmol) and 4-tert-butylphenylisocyante (200 μL, 0.5 mmol) were added at 0° C. The reaction mixture was warmed to room temperature and stirred for 1 h. Upon removal of the solvent, the residue was purified with column chromatography (silica gel, EtOAc:methanol 20:1) to give the cyclohexanone protected compound of Formula VI (294 mg, 95%) as a white solid, which was treated with HCl (1.0 mL, 4 M in dioxane) in MeOH (3.0 mL) for 12 h. After removal of the solvent, the solid was washed with ethyl acetate (3×3 mL) to give the compound of Formula VI as a white powder (265 mg, 92%). ¹H NMR (400 MHz, d₆-DMSO): δ 8.38-8.19 (m, 2H), 7.29-7.18 (m, 4H), 5.40 (br, 2H), 4.78-4.72 (m, 1H), 4.36-4.34 (m, 1H), 4.01-3.98 (m, 1H), 3.91-3.89 (m, 1H), 3.81-2.99 (m, 7H), 2.38-2.31 (m, 2H), 198-1.88 (m, 3H), 1.40-1.16 (16H). HRMS (ESI) [M+H]⁺ Calculated for C₂₈H₄₃N₈O₃⁺: 539.3453. Found: 539.3450.

### Example 4

The compounds of Reaction Scheme II were synthesized as follows.

Compound 19.

Compound 18 was prepared from D-ribose as described in Tetrahedron 2007, 63, pp 9836-9841, which is incorporated by reference herein in its entirety. A solution of compound 18 (1.53 g, 3.6 mmol) in CH₂Cl₂ (13 mL) was degassed followed by addition of 2^{nd} generation Grubbs' catalyst (118 mg). The reaction mixture was refluxed overnight. Additional 2^{nd} generation Grubbs' catalyst (59 mg) was added to increase the yield and the reaction was continued for 6 h. Upon removal of the solvent, the residue was purified with column chromatography (silica gel, EtOAc/hexanes 1:6) to give compound 19 (1.45 g, 95%) as a colorless oil. ¹H NMR (400 MHz, CDCl₃): δ 7.75-36 (m, 10H), 5.98-5.97 (m, 1H), 5.77-5.75 (m, 1H), 5.36-5.34 (m, 1H), 4.55 (d, J=4.8 Hz, 1H), 4.01 (d, J=9.6 Hz, 1H), 3.70 (d, J=9.6 Hz, 1H), 3.23 (br, 1H), 1.36 (s, 3H), 1.28 (s, 3H), 1.08 (s, 9H).

Compound 20.

To a solution of compound 19 (4.42 g, 10.4 mmol) in 40 mL of N,N-dimethylformamide (DMF) was added 4 Å molecular sieve (4.4 g) and pyridinium dichromate (PDC, 8.4 g, 20.8 mmol). The reaction mixture was stirred for 12 h at room temperature. The precipitation was filtered off and washed with EtOAc. The filtrate was washed with water (20 mL) and brine (20 mL), dried over Na2SO4, concentrated under reduced pressure, and purified with column chromatography (silica gel, EtOAc/hexanes 1:6) to give compound 20 as a colorless oil (3.83 g, 87%). ¹H NMR (400 MHz, CDCl₃): δ 7.75-36 (m, 10H), 6.33 (s, 1H), 4.96 (d, J=5.2 Hz, 1H), 4.70 (d, J=18.4 Hz, 1H), 3.50 (d, J=18.4 Hz, 1H), 3.49 (d, J=5.2 Hz, 1H), 1.35 (s, 3H), 1.34 (s, 3H), 1.07 (s, 9H).

Compound 21.

To a solution of compound 20 (1.77 g, 4.2 mmol) in MeOH (20 mL) was added CeCl₃.7H₂O (1.32 g, 3.5 mmol) and NaBH₄ (311 mg, 8.4 mmol) at 0° C. The reaction mixture was warmed to room temperature and stirred for 1 h. Upon removal of the solvent, water (10 mL) was added and pH adjusted to 5 with acetic acid. The product was extracted with EtOAc (3×20 mL). The combined organic phases were washed with brine (10 mL), dried over Na₂SO₄, concentrated under reduced pressure, and purified with column chromatography (silica gel, EtOAc/Hexanes 1:4) to give compound 21 as a colorless oil (1.75 g, 98%). ¹H NMR (400 MHz, CDCl₃): δ 7.68-7.37 (m, 10H), 5.82 (s, 1H), 4.74 (t, J=5.6 Hz, 1H), 4.55-4.53 (m, 1H), 4.38 (d, J=15.2 Hz, 1H), 4.28 (d, J=15.2 Hz, 1H), 1.35 (s. 3H), 1.33 (s, 3H), 1.06 (s, 9H).

Compound 22.

To a solution of compound 21 (545 mg, 1.28 mmol), 6-chloropurine (297 mg, 1.92 mmol) and Ph₃P (671 mg, 2.56 mmol) in THF (16 mL) was added disopropyl azodicarboxylate (DIAD, 528 μL, 2.56 mmol) dropwise at 0° C. The reaction mixture was allowed to warm to room temperature and stirred overnight. Upon removal of the solvent, the residue was subjected to column chromatography (silica gel, EtOAc/Hexanes 1:3) to give compound 22 as a colorless oil (681 mg, 95%). ¹H NMR (400 MHz, CDCl₃): δ 8.88 (s, 1H), 7.90 (s, 1H), 7.67-7.37 (m, 10H), 5.83 (s, 1H), 5.64 (s, 1H), 5.28 (d, J=5.2 Hz, 1H), 4.72 (d, J=5.2 Hz, 1H), 4.48 (dd, J=12.4, 6.8 Hz, 2H), 1.41 (s, 3H), 1.37 (s, 3H), 1.08 (s, 9H).

Compound 23.

Compound 22 (616 mg, 1.1 mmol) was dissolved in 7 M NH₃ in MeOH (3 mL) and heated to 100° C. in a sealed pressure flask overnight. After cooling, the solvent was removed and the residue was dissolved in THF (6 mL). Tetrabutylammonium fluoride (1.6 mmol, 1.6 mL as a 1 M THF solution) was added and stirred for 2 h at room temperature. Upon removal of solvent, the residue was subjected to column chromatography (silica gel, 5% methanol in ethyl acetate) to give compound 23 as a white foam (313 mg, 93%). ¹H NMR (400 MHz, d₆-DMSO): δ 8.18 (s, 1H), 7.97 (s, 1H), 7.01 (br, 2H), 5.74 (s, 1H), 5.42 (s, 1H), 5.38-5.37 (m, 1H), 5.03 (br, 1H), 4.68-4.67 (m, 1H), 4.18 (s, 2H), 1.39 (s, 3H), 1.28 (s, 3H).

### 1-(3-((((3R,4S,5R)-3-adenosyl-4,5-dihydroxycyclopent-1-en-1-yl)methyl)-(isopropyl)amino)propyl)-3-(4-(tert-butyl)phenyl)urea (Compound of Formula VII)

Using steps xii and xviii (Reaction Scheme I) described in the making of compound of Formula VI, compound 23 (487.0 mg, 1.2 mmol) was converted to give compound 7 as a white powder (440 mg, 60%). ¹H NMR (400 MHz, D₂O): δ (rotamer: 1:1) 8.17, 8.16 (s, 1H), 8.10, 8.06 (s, 1H), 7.04-6.79 (m, 4H), 6.33, 6.34 (s, 1H), 5.44-5.39 (m, 1H), 4.28-4.19 (m, 1H), 4.06-3.97 (m, 1H), 3.83-3.58 (m, 2H), 3.52-3.48 (m, 1H), 3.43-3.9 (m, 1H), 3.26-3.18 (m, 3H), 1.98-1.86 (m, 2H), 1.34-1.24 (m, 6H), 1.05 (s, 9H). HRMS (ESI) [M+H]⁺ Calculated for C₂₈H₄₁N₈O₃⁺: 537.3296. Found: 537.3292. Compound 23 was transformed to the compound of Formula VII with an overall yield of 29.2% from D-ribose.

### 1-(3-((((1 S,2R,3S,4R)-4-adenosyl-2,3-dihydroxycyclopentyl)methyl)(isopropyl)-amino)propyl)-3-(4-(tert-butyl)phenyl)urea (compound epi-6)

To a solution of compound of Formula VII (35 mg, 0.057 mmol) in MeOH (4 mL) was added 10% Pd/C (5 mg). The reaction mixture was stirred at room temperature with a hydrogen balloon for 2 h. The reaction mixture was filtered through a 0.2 μm syringe filer and washed with methanol. The filtrate was concentrated under reduced pressure to afford compound epi-6 as a white solid (32 mg, 91%). ¹H NMR (400 MHz, D₂O): δ (rotamer: 1:1) 8.35, 8.34 (s, 1H), 8.30, 8.29 (s, 1H), 7.39 (d, J=10.4 Hz, 1H), 7.18 (d, J=10.4 Hz, 1H), 4.78-4.76 (m, 1H), 3.63-3.57 (m, 2H), 3.37-2.98 (m, 7H), 1.99-1.91 (m, 1H), 1.82-1.78 (m, 2H), 1.38-1.18 (m, 17H). HRMS (ESI) [M+H]⁺ Calculated for C₂₈H₄₃N₈O₃⁺: 539.3453. Found: 539.3449. Hydrogenation of compound of Formula VII from the less hindered up side of the cyclopentene ring gave compound epi-6 in 91% yield.

### Example 5

DOT1L inhibitors were prepared and enzyme inhibition assays were conducted as described in Example 1. Some embodiments herein used a ligand and/or a structure based approach using a comparative analysis between the X-ray structure of DOT1L/SAM and those of other HMTs in complex with SAH.

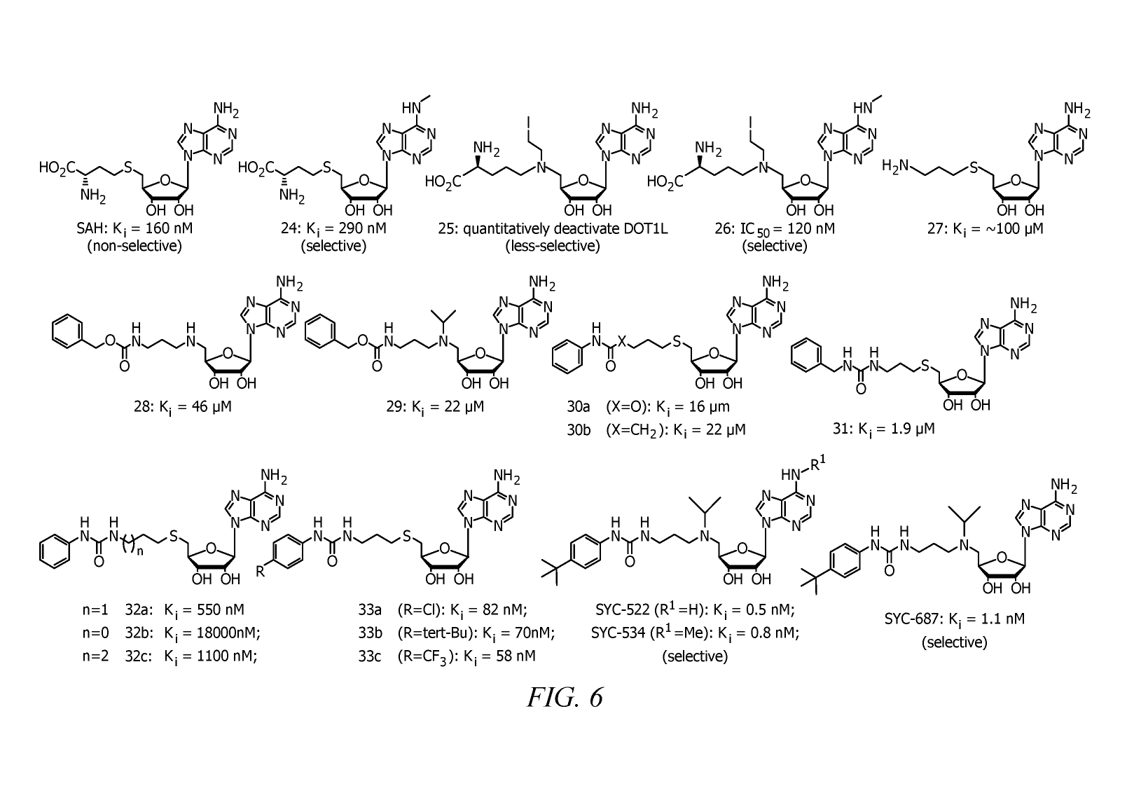

N6-substituted SAH analogs such as compound 24 of FIG. 6 were thus designed to selectively target DOT1L, since these N6-substitutents were predicted to disrupt key ligand-protein interactions with other HMTs, but not with those with DOT1L.

Indeed, compound 24 (or N6-methyl-SAH) was found to be a potent inhibitor of DOT1L with a Kᵢ of 290 nM, while it was found to be inactive against a panel of other HMTs, showing a high selectivity for DOT1L.

A mechanism based approach was also applied to find more potent inhibitors, such as compounds 25 and compound 26, which may form an aziridinium intermediate that mimics the methyl-sulfonium moiety of SAM, these compounds were also found to be potent DOT1L inhibitors.

Compounds were also developed to remove the amino acid moiety in SAH (such as compound 24) to improve the cell membrane permeability while maintaining DOT1L activity, though compound 27 (or decarboxy-SAH) for example was found to be almost inactive against DOT1L, and many other 5′-substituted adenosine compounds are inactive or only weakly active.

Carbamate compound 28 (FIG. 6), which is a Z-protected intermediate for synthesizing a 5′-amino analog of 3, was surprisingly found to be a better inhibitor than compound 27. Due to the more hydrophobic nature of its side-chain, medicinal chemistry based on compound 28 was also performed. Adding an additional isopropyl group onto 5′-N resulted in compound 29 showing an increased activity. Reversing the carbamate functionality or changing to an amide resulted in compounds 30a and 30b with improved activity. About a 25-fold activity enhancement was observed for the urea compound 31 with a Kᵢ of 1.9 μM, as compared to the carbamate 28. The phenyl urea compound 32a with a Kᵢ of 550 nM was found to be superior to the benzyl analog 31. Thus these embodiments indicate that both —NH— moieties of the urea group are important, with each one offering about a 25-fold to greater than about 50-fold activity enhancement, as compared to less favored —O— and —CH₂— groups (e.g., compound 31 vs. compound 28, as well as compound 32a vs. compounds 30a/b). These two —NH— may serve as H-bond donors to increase the binding affinity to DOT1L. In addition, weaker activities of compounds 32b and 32c suggest the optimal linker between the urea group and the 5′-position may be —CH₂CH₂CH₂— or analogs thereof. Additional structure-activity relationship (SAR) study revealed that an appropriate 4-substituent, such as 4-CI, 4-tert-Bu and 4-CF₃ in compounds 33a, 33b, and 33c, is able to further improve the activity by up to 10-fold. Finally, replacing the —S— in 33b with an —N(i-Pr)—, which shows improved activity in compound 29, led to the more potent DOT1L inhibitors SYC-522 and -534 with Kᵢ values of 0.5 and 0.8 nM. The large activity boost (as compared to that of compound 33b) may be due to coordinated protein conformational changes induced by co-binding of the N-(4-tert-butylphenyl)urea side-chain and the —N(i-Pr)— group.

Urea-containing DOT1L inhibitors such as SYC-522 and EPZ004777 exhibit a very high enzyme selectivity of >1,200 against a panel of eight HMTs. X-ray crystallographic studies indicated this high enzyme selectivity is due to a large protein conformational change induced by the hydrophobic urea side-chain of the inhibitor, these compounds possess selective activity against MLL-leukemia showing the promise to be used to treat MLL leukemia. However, a problem with these known ribose-containing DOT1L inhibitors in animal studies is their metabolic instability, resulting in a poor PK, e.g., a short half-life in plasma. Many human enzymes can recognize adenosine or deaza-adenosine moiety, leading to rapid cleavage of adenine and/or 5′-substituent. Poor PK is responsible for the weak in vivo activity of EPZ004777 in a MV4-11 MLL leukemia mouse model. More metabolically stable DOT1L inhibitors are therefore needed.

Further embodiments herein provide a solution to these limitations by replace the ribofuranose ring of these compounds with a 5-membered carbocycle. Compound SYC-687 (FIG. 6) with a cyclopentane ring was efficiently synthesized in 20 steps with an overall yield of 19%, starting from readily available D-ribose as described in Med. Chem. Commun. 2013, 4, pp 822-826, which is incorporated by reference herein in its entirety. SYC-687 shows a DOT1L inhibitory activity with a Kᵢ of 1.1 nM. However, this cyclopentane-containing DOT1L inhibitor was found to exhibit high metabolic stability (CLint=0.36 μL/min/mg protein) in human plasma and liver microsomes, the latter of which are mainly responsible for drug metabolism. Like its deaza-analog EPZ004777, the ribose-containing inhibitor SYC-522 can be quickly degraded in the presence of human liver microsomes with an intrinsic clearance value (CLint) of 24 μL/min/mg protein. Cyclopentane-containing DOT1L inhibitors are metabolic stability and represent a direction for future drug development.

### Example 6

### The Importance of H3K79 Methylation in DOT1L in Breast Cancer

Since the biological functions of DOT1L (or H3K79 methylation) in cancers other than MLL-leukemia had not been explored, DOT1L-specific inhibitor SYC-522 was tested herein against a panel of cell lines and found to be selective activity against certain TNBCs. Table 1 displays the results of an MTT assay of antiproliferation activity (μM) against TNBCs and other cells.

TABLE 2

| | MDA- | | | | | | | | |
|---|---|---|---|---|---|---|---|---|---|
| | MB231 | | BT549 | | MB468 | HCC70 | NB4 | A549 | WI-38 |
| | 3-day | 15-day | 3-day | 15-day | 15-day | 15-day | 15-day | 15-day | 15-day |
| SYC-522 | >100 | 1.2 | >100 | 1.8 | 36 | 18 | >100 | >100 | >100 |

As shown in Table 2, SYC-522 exhibited strong activity (EC50: 1.2, 1.8 μM) against BC cells MDA-MB231 and BT549 during a 15-day treatment, while it was weakly active against basal-like TNBC MDA-MB468 and HCC70 (EC50: 36, 18 μM), or inactive against other cancer cells (i.e., non-MLL leukemia NB4 and lung cancer A549) and normal fibroblast WI-38 cells. Also, the activity of SYC-522 is not due to cytotoxicity (inactive in a 3-day treatment for all cell lines). The characteristic slow action of histone methylation inhibitors is likely due to the long time required for cellular events that lead to cell growth arrest, including blocked histone methylation (maximal effect shown on about day 4), followed by decreased levels of mRNA expression for the targeted genes, as well as ultimately the depletion of the gene products (proteins) key to the cell proliferation. Selective activity of the DOT1L-specific inhibitor against CLBC cells is of interest, since this suggests H3K79 methylation plays an important role in cancer biology of CLBC.

Embodiments herein also characterize how SYC-522 affects CLBCs.

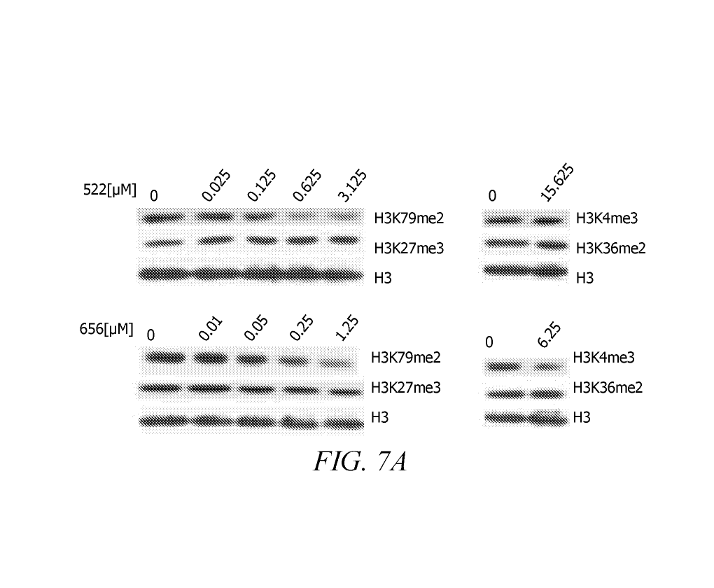

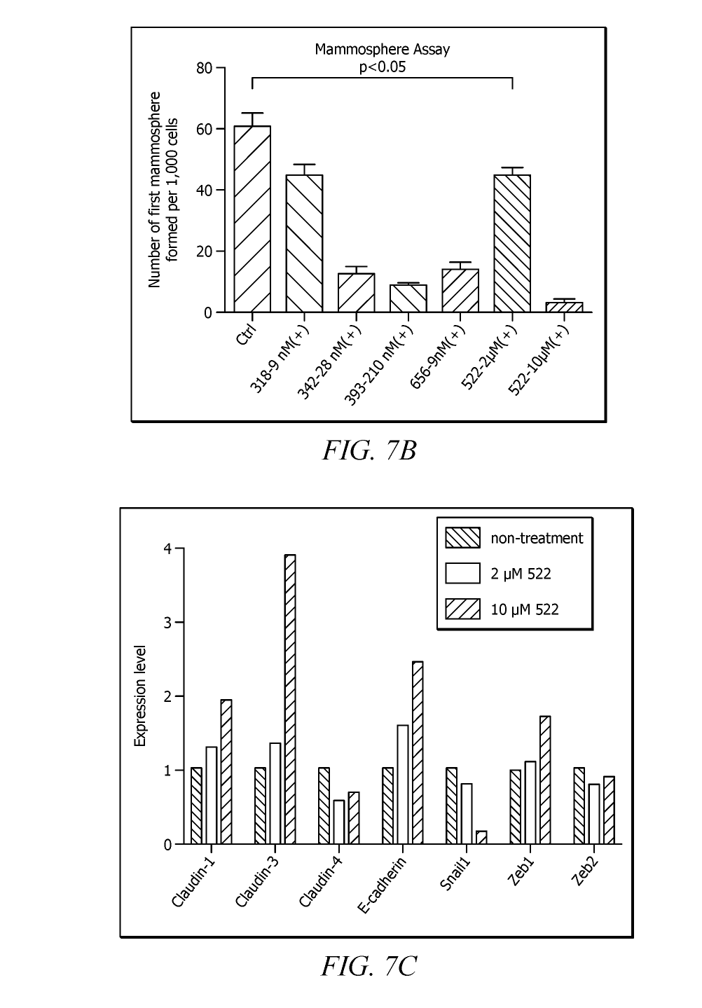

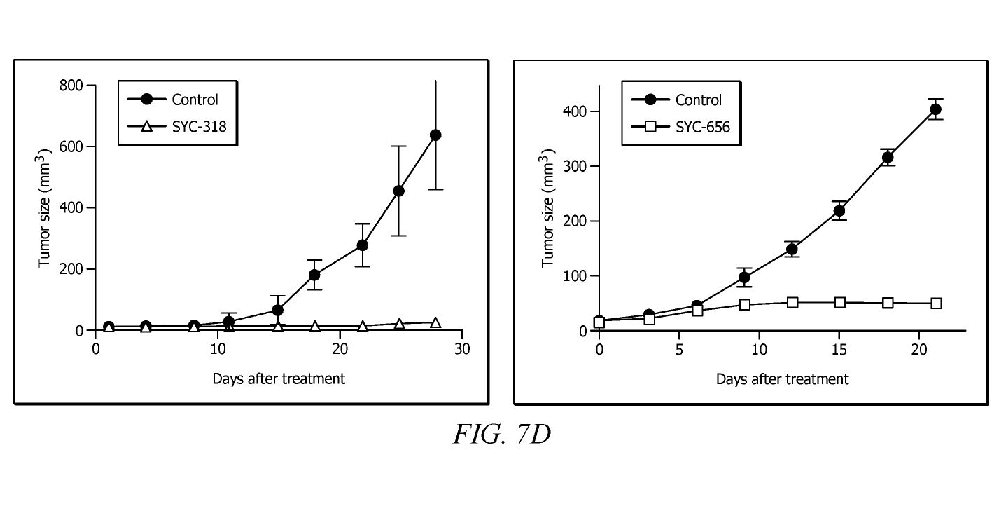

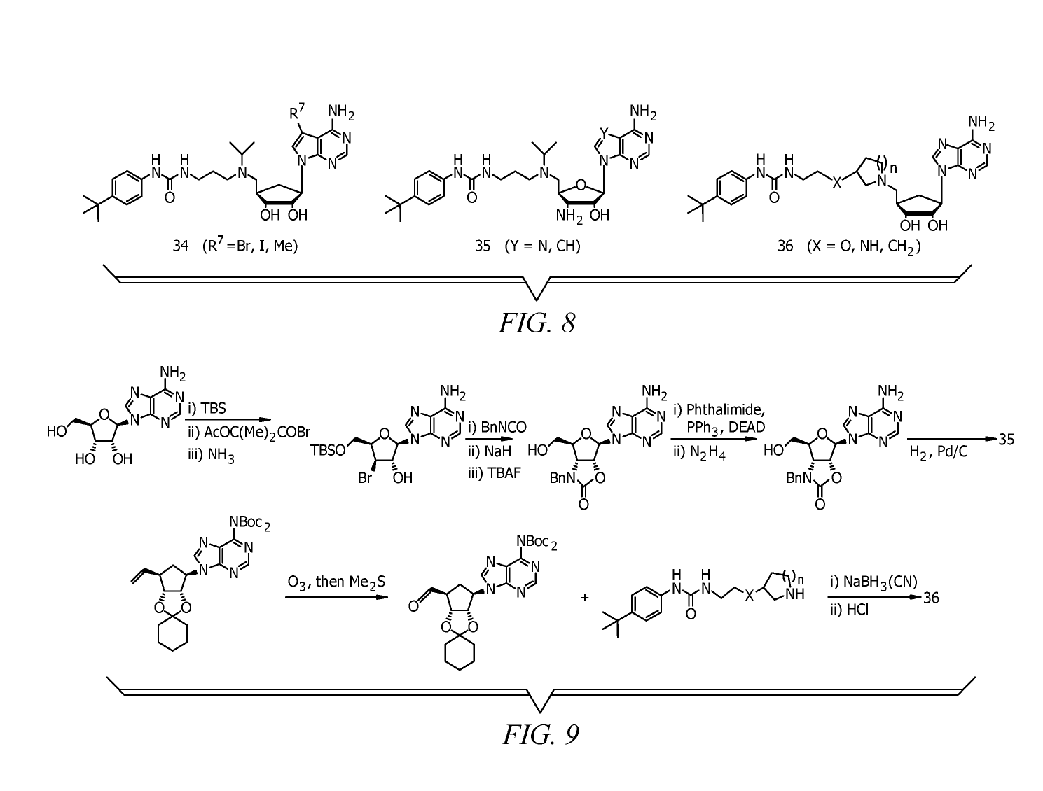

First, SYC-522 selectively inhibited histone methylation at H3K79 with an EC50 of ˜150 nM in MDA-MB231 cells (FIG. 7A), similar to the activity in MV4-11 cells. This, combined with its non-cytotoxic nature, suggests that H3K79 methylation targeted genes/proteins are critical to MDA-MB231.

Second, using FACS with an Annexin-V assay, SYC-522 was found to cause negligible apoptosis of MDA-MB231 at 2 μM for a 10-day treatment and 18.4% apoptosis at 10 μM.

Third, a mammosphere formation assay was used as the first step towards testing the activity of SYC-522 against breast CSC. As such, in one embodiment, 1,000 MDA-MB231 cells/well were cultured in 1 mL of a serum-free medium (Mammocult Basal medium, 10% Mammocult supplements, 10 μM hydrocortisone and 0.004% Heparin) with or without compounds for 7 days in a 24 well non-attachment microplate (Corning). At 2 μM, SYC-522 exhibits ˜30% inhibition and may need longer treatment to show more activity. At 10 μM, it can inhibit >90% mammosphere formation (FIG. 7B). These results indicate that SYC-522's activity on MDA-MB231 is not due to apoptosis, but likely attributed to its inhibition of self-renewal ability and induction of differentiation of CSC.

Fourth, qPCR was used to find if treatment with SYC-522 affects gene expression of cell-cell adhesion and EMT genes. FIG. 7C shows that SYC-522 causes considerable, dose-dependent increases (as much as 3.9-fold) of the expression of claudin-1, -3 and E-cadherin as well as a large decreased expression of Snail1, while Zeb1 and Zeb2 expression remains unchanged or slightly increased. While further qPCR as well as a comprehensive microarray experiments will be used to further investigate H3K79 inhibition mediated gene expression changes, the current results indicate that SYC-522 may significantly promote cell-cell adhesion (which may inhibit cancer cell invasion/migration) by up-regulating claudins and E-cadherin. Further embodiments of the methods herein described may be used to probe SYC-522's activity on EMT.

### Example 7

### DOT1L Inhibitor Design

Embodiments herein described were used to identify two series of compounds that possess selective activity against CLBC by potently inhibiting H3K79 methylation. Rational inhibitor design and medicinal chemistry were used to develop DOT1L and SAHH inhibitors with improved activity and/or PK properties, as compared to known inhibitors of DOT1L.

Binding Mode Approach to Design.

A common feature of the binding mode of DOT1L inhibitors is the adenosine (or deaza-adenosine) moiety, which is recognized by five H-bonds with DOT1L, including 1) adenine 6-NH2 with Asp222; 2) adenine N5 with Phe223; 3) adenine N3 with Lys187; 4/5) ribose 2′, 3′-diol with Glu186. Compounds with potentially improved activity and stability, were designed herein. Compound 34 was based on cyclopentane-containing inhibitor SYC-687, which is metabolically stable, but has a less enzyme potency (Kᵢ=1.1 nM). Adding a 7-substituent such as —Br is known to improve the binding affinity. For example, 7-Br-SAH is ˜8× more active than SAH. Compound 34 with a 7-substituent is expected to be more potent than SYC-687. The 3′-NH2 of compound 35 may provide enhanced binding by more electrostatic/H-bond interactions with Glu186, but also be may be more stable. Cyclic sidechains in compound 36 were designed to have reduced rotatable bonds, providing enhanced binding affinity.

Fragment Based Design.

SYC-522 contains two fragments: adenosine and 4-tert-butylphenyl-urea linked by —CH2CH2CH2-. 4-tert-butylphenyl-urea is known to provide high selectivity as well as affinity, while adenosine is metabolically unstable. Embodiments herein were used to design molecules with a suitable replacement moiety for the adenosine fragment. The method developed comprises: (i) screening several fragment compound libraries to find fragment molecule(s) having a Kᵢ of <500 μM against recombinant human DOT1L, using a high-throughput screen with Perkin-Elmer MicroBeta2 scintillation counter with a 96-well harvesting system; (ii) confirming identified hits with enzyme kinetics, and performing X-ray crystallographic study to find the fragment molecules that occupy the adenosine binding site of DOT1L; (iii) synthesizing molecules linking the fragments with the 4-tert-butylphenyl-urea moiety; and (iv) using medicinal chemistry to find compounds with improved activity. An embodiment of such a synthetic mechanism is provided in FIG. 9.

In Vitro Biological Activity Evaluations.

Compounds could be tested for their inhibitory activities against recombinant enzyme human DOT1L or SAHH. DOT1L inhibitors were further evaluated for their inhibition against other HMTs for their selectivity. Potent and selective inhibitors could be further tested for their activity against H3K79 methylation, and proliferation of a panel of breast cancer cell lines including three CLBCs: MDA-MB231, 157 and BT549, two basal-like TNBC: MDA-MB468 and HCC70; and ER+MCF-7. Various other molecular biology studies using SYC-522 could be performed to characterize inhibitor activity on MDA-MB231.

HMT Enzyme Selectivity.

Very potent DOT1L inhibitors designed by embodiments described herein, could be tested for their selectivity against two SET-domain HKMTs (G9a and SUV39H1) and two histone arginine methyltransferases PRMT1 and CARM1.

Cellular Histone Methylation Inhibition Using Western Blot.

MDA-MB231 or other CLBC cells in the exponential growth phase were incubated in the presence of increasing concentrations of a compounds (0.025-15.625 μM). Cells (2×10⁶) were harvested on day 4 and histones were extracted with the EpiQuik™ total histone extraction kit (Epigentek) according to the manufacturer's protocol. Equal amounts of histones (2 μg) were separated with SDS-PAGE and transferred to a piece of PVDF membrane. The blots were incubated with primary antibodies against a specific histone methylation and Histone H3 (Cell Signaling), followed by secondary antibody (anti-rabbit IgG) coupled with horseradish peroxidase (HRP), and detected with Supersignal West Dura substrate (Thermo Scientific). Potent and selective inhibitors of DOT1L and SAHH could be tested for their ability to block H3K79Me2, H3K4Me3, H3K9Me3, H3K27Me3, H3K36Me2 and H4K20Me3.

Human Cell Cytotoxicity Assay.

Highly potent and selective compounds could be incubated for 3 days with three human cell lines, i.e., WI-38 (normal fibroblast), NB4 (non-MLL leukemia) and A549 (lung cancer), to evaluate the potential toxicity of compounds designed and synthezised by embodiments described herein, using a standard MTT assay. Compounds that have potent and selective activity against DOT1L or SAHH but show no or negligible cytotoxicity in these assays could be chosen to be further tested for anti-proliferative activity against CLBC as well as other subtypes of breast cancer cells.

In Vitro Anti-Proliferative Activity Against BC and Other Subtypes of Breast Cancer Cells.

The compounds designed herein could be tested for their activities for a 3-day and 15-day treatment against three CLBC (MDA-MB231, 157 and BT549), two basal-like TNBC (MDA-MB468 and HCC70), one ER+ MCF-7 and one HER2+ SK-BR-3 cell lines. MDA-MB157 and SK-BR-3 could be purchased from ATCC. In some embodiments, the 3-day drug treatment could follow the same protocol as the cytotoxicity assay described above. For 15-day treatment, after every 3 days the culture media containing a compound designed herein could be aspirated and cells could be washed with PBS and trypsinized. 10⁵ cells from each well could be added to the corresponding well of a new 96-well plate for the next round of culture. Cell viability after 15 days could be evaluated using the standard MTT assay. Data from each well could be imported into Prism 5.0 and EC50 values could be calculated from the dose-responsive curves.

In Vitro Activity to Inhibit MDA-MB231 Invasion.

Matrigel coated microplate with a 8 μm pore size could be used to determine if SYC-522 blocks the in vitro migration/invasion of MDA-MB231 cells, following well known protocols. MDA-MB231 cells could be treated with SYC-522 (at 2 and 10 μM) for 10 days and could be trypsinized. After washing, 5×104 cells/well in ˜1 mL of DMEM (with SYC-522) could be added to the inserts of the 24-well Matrigel microplate. These inserts could be placed on top of the well containing 5% fetal bovine serum in DMEM as chemoattractant. After incubation for 6 h, number of cells that pass through the Matrigel membrane into the bottom chamber could be counted. Non-treated MDA-MB231 cells could be used as control.

Investigation of Possible Mechanisms.

In one embodiment, the DOT1L-specific inhibitor SYC-522 shows selective activity against claudin-low TNBC cells by a different mechanism from known chemotherapeutic drugs. It is therefore of interest to investigate possible mechanisms of action of SYC-522 on MDA-MB231. SYC-522 specifically blocks H3K79 methylation, reverses certain dysregulated gene expression of claudins, E-cadherin and EMT that are characteristic to CLBC, and inhibits mammosphere formation suggesting activity against cancer stem cells, thus embodiments are described herein to further characterize the biological activity of SYC-522/H3K79 methylation inhibition: (1) treating MDA-MB231 with several shRNA targeting DOT1L available from Invitrogen (Grand Island, N.Y.) to see if DOT1L knock-down will produce the same or similar effect as SYC-522; (2) testing whether SYC-522 induces differentiation of CSC to have reduced population of CSC with CD44+/CD24−/low/ALDH+ and increased population exhibiting epithelial (expressing ESA) and/or myoepithelial (expressing CD10) using commercially available fluorescence-labeled antibodies monitored by FACS; (3) performing a comprehensive microarray analysis to understand the global gene expression changes caused by H3K79 methylation inhibition, from which groups of genes for a certain cellular function or signaling pathway that mostly affected by SYC-522 could be identified; and/or (4) using qPCR and Western blot to confirm important gene changes.

In Vivo Antitumor Activity Testing.

In vivo PK/Tox properties of compounds designed and developed by embodiments described herein could be tested for antitumor and anti-metastasis activities in two CLBC mouse models. Paclitaxel (Taxol), one of the most often used chemo-drugs for breast cancer, could be included in in vivo studies as a comparison. Prior studies have shown that although a chemodrug showed potent in in vivo growth inhibition against TNBC models, it does not affect CSC and can even cause the CSC population to be enriched (since it kills more non-stem-like breast cancer cells).

PK/Tox Testing of Inhibitors (Designed by Embodiments Described Herein) in Animal Studies.

In vitro PK properties could be tested, i.e., plasma protein binding, Caco-2 (intestine epithelial cells) bidirectional permeability, metabolic stabilities in plasma and liver microsome and inhibition of cytochrome P450. Based on the results, ˜5 compounds could be further selected to be tested for their in vivo PK/Tox. Further, the MTD (most tolerated dose) for an intraperitoneal (i.p.) injection in nude mice could be found; and the PK parameters (e.g., plasma elimination half life, area-under curve or AUC, etc.) could be assessed using a non-toxic dose (e.g., ½ of MTD). Data from these PK/Tox studies could be used to determine the initial dosages and administration method for further in vivo testing.

In Vivo Activity Against MDA-MB231 Xenograft Mouse Model.

Compounds designed herein, with satisfactory PK parameters (t½>3 h with a high AUC) could be selected to be tested for their antitumor activity in the MDA-MB231 mouse model. Soluble compounds could be dissolved in sterile PBS for injection. Non-soluble, compounds could be first dissolved in a minimal amount (<1% final volume) of DMSO and then could be diluted with sterile PBS. An approved surfactant of <1% of final volume (e.g., Cremophor-EL from Sigma) could be added to assist solubilization of compounds. Drug treatment could be started ˜10 days after tumor transplantation when small (3-5 mm in diameter) tumors are palpable. Tumor bearing mice could be randomly separated (e.g., randomized) into control and treatment groups with 10 mice/group and could be treated with these compounds for 14 or 28 days. The unusually long treatment periods are due to the in vitro results showing the slow action of these epigenetic inhibitors. Tumor sizes could be measured every three days and estimated by using the formula (length×width²/2). By the end of the treatment, all mice could be sacrificed and cells freshly separated from tumor tissues could be analyzed to find if in vivo treatment of these compounds may: (1) suppress the methylation at H3K79 and other histone sites by western blot; (2) reduce the population of breast CSC with CD44+/CD24−/low cell markers in the tumor by FACS; and/or (3) inhibit the mammosphere-forming ability of these cells when cultured in a drug-free medium. The remaining part of the tumors could be fixed with paraformaldehyde and sections could be immunefluorescence stained with antibodies against claudins, EMT and other genes of interest to find if the drugs affect these biomarkers in vivo. The digital images (from >20 high power fields) could be analyzed with an image analysis software ImageJ to quantitate the results. The relative abundance in the treatment groups with respect to the control could be analyzed using SigmaStat.

In Vivo Activity Against the Metastatic MDA-MB231-LM3.3 Mouse Model.

10⁶ cells could be injected into mammary fat pads of nude mouse (n=10 per group). When tumors grow to 3-5 mm in diameter, drug treatment and tumor size measurement could be performed as described above. At the experimental end point, mice could be euthanized and their left lung could be used for luciferase assay to quantitate the relative metastasis index for the luciferase-labeled LM3.3 cells. Specifically, lung tissue could be frozen in liquid N₂, tissue powder could be prepared under frozen condition, tissue protein could be extracted by using a lysis buffer, and 100 μg protein could be used for luciferase assay. The right lung of these mice could be fixed in 4% paraformaldehyde, embedded in paraffin and sectioned for immunostaining for GFP to specifically observe lung metastasis derived from GFP-labeled LM3.3 xenograft tumors in the fat pads. Three coronal sections with at least 100 μm interspace to each other could be immunostained. On the lung images, the areas of GFP-positive LM3.3 tumor cells and the total lung areas could be measured using a histological analysis software and the metastasis index could be calculated by dividing the tumor area with total lung area. This measurement could be performed in all mice to quantitatively evaluate the effects of all treatments on lung metastasis.

Compounds disclosed herein that inhibit both primary xenograft growth and metastasis could be further examined to determine if the inhibition of metastasis is caused by the drug or the greatly reduced primary tumor burden, using an IV LM3.3 model, as described above. 5×10⁵ LM3.3 cells could be injected into the mouse tail vein (n=10 for each group), and the lung metastasis could be allowed to develop for 7 days. Drug treatment could be started on day 8. At the experimental end point, luciferase activity in the lung tissue lysates and GFP immunostaining could be performed as described above to quantify the extent of lung metastasis. Data could be statistically analyzed by One-Way ANOVA.

Thus, it is disclosed herein that histone H3-lysine79 (H3K79) methyltransferase DOT1L plays a crucial role in many BC. Exploring a database containing >1000 BC and normal tissues, it was found that DOT1L highly correlates with BC as well as overexpression of many BC oncogenes. A gene expression analysis was performed, wherein results from a gene expression database were compiled, 1,032 normal breast and breast cancer tissues, wherein DOT1L was found to highly correlate (p<0.001) with breast cancer and overexpression of all of the 23 oncogenes in the left panel of PAM50 gene set that are important for breast cancer. A potent and specific DOT1L inhibitor SYC522, which is inactive against other cancer/normal cells, exhibits selective activity (EC50 ˜1 μM) against BC cell lines (e.g., MDA-MB231, BT549 and MCF-7). Mechanistically, SYC522 is non-cytotoxic, inhibits specifically H3K79 methylation (H3K79me), blocks the self-renewal ability and induces differentiation of BCSC, and reverses the expression of certain disregulated genes involved in metastasis. Thus, BCSC could be highly dependent on certain H3K79me targeted genes, while normal cells can tolerate DOT1L inhibition. Rational inhibitor design, medicinal chemistry, and X-ray crystallography was thus used to develop potent inhibitors of H3K79me, which are biological activity against BCSC, (and in one embodiment active in a metastatic MDA-MB231 mouse model). Such compounds could be less toxic drug candidates, compared to known compounds that are effective against BCSC and metastasis.

While embodiments of the invention have been shown and described, modifications thereof can be made by one skilled in the art without departing from the spirit and teachings of the invention. The embodiments described herein are exemplary only, and are not intended to be limiting. Many variations and modifications of the invention disclosed herein are possible and are within the scope of the invention. Where numerical ranges or limitations are expressly stated, such express ranges or limitations should be understood to include iterative ranges or limitations of like magnitude falling within the expressly stated ranges or limitations (e.g., from about 1 to about 10 includes, 2, 3, 4, etc.; greater than 0.10 includes 0.11, 0.12, 0.13, etc.). For example, whenever a numerical range with a lower limit, Rₗ, and an upper limit, Rᵤ, is disclosed, any number falling within the range is specifically disclosed. In particular, the following numbers within the range are specifically disclosed: R=Rₗ+k*(Rₙ−Rₗ), wherein k is a variable ranging from 1 percent to 100 percent with a 1 percent increment, i.e., k is 1 percent, 2 percent, 3 percent, 4 percent, 5 percent, . . . 50 percent, 51 percent, 52 percent, . . . , 95 percent, 96 percent, 97 percent, 98 percent, 99 percent, or 100 percent. Moreover, any numerical range defined by two R numbers as defined in the above is also specifically disclosed. Use of the term “optionally” with respect to any element of a claim is intended to mean that the subject element is required, or alternatively, is not required. Both alternatives are intended to be within the scope of the claim. Use of broader terms such as comprises, includes, having, etc. should be understood to provide support for narrower terms such as consisting of, consisting essentially of, comprised substantially of, etc.

Accordingly, the scope of protection is not limited by the description set out above but is only limited by the claims which follow, that scope including all equivalents of the subject matter of the claims. Each and every claim is incorporated into the specification as an embodiment of the present invention. Thus, the claims are a further description and are an addition to the embodiments of the present invention. The discussion of a reference in the Detailed Description of the Embodiments is not an admission that it is prior art to the present invention, especially any reference that may have a publication date after the priority date of this application. The disclosures of all patents, patent applications, and publications cited herein are hereby incorporated by reference, to the extent that they provide exemplary, procedural or other details supplementary to those set forth herein.

1. A compound of Formula I, a pharmaceutically acceptable salt thereof, a prodrug thereof, or combinations thereof: 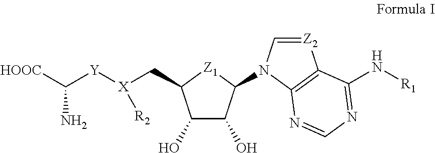
wherein R₁ is H, methyl, or benzyl;
R₂ is 2-cyanoethyl, 2-methoxycarbonylethyl, or 2-iodoethyl;
X is N or S; wherein if X=S, R₂=O;
Y is C₃ or C₄;
Z₁ is O, S, N, or CH₂; and
Z₂ is N or CR₄, wherein R₄ is a halogen, alkyl, aryl or a 5- or 6-membered heterocycle; and
wherein said compound is selective for DOT1L Methyl Transferase.

2. A compound of Formula II, a pharmaceutically acceptable salt thereof, a prodrug thereof, or combinations thereof: 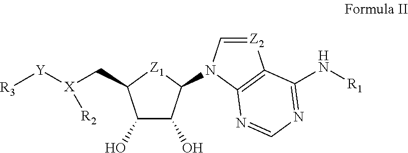
wherein R₁ is H, alkyl, or benzyl;
R₂ is H, 2-cyanoethyl, 2-methoxycarbonylethyl, methyl, 2-iodoethyl, ethanol, butyl, or benzyl carbamate;
X is N, C, or S; wherein if X=S, R₂=O; and wherein if X=C, R₂ is also equal to R₃ or R₁, and Y is also equal to R₁, R₂ or R₃;
Y is C₁, C₂, C₃ or C₄;
Z₁ is O, S, N, or CH₂;
Z₂ is N, or CR₄, wherein R₄ is a halogen, alkyl, aryl or a 5- or 6-membered heterocycle;
R₃ is H or selected from the following: 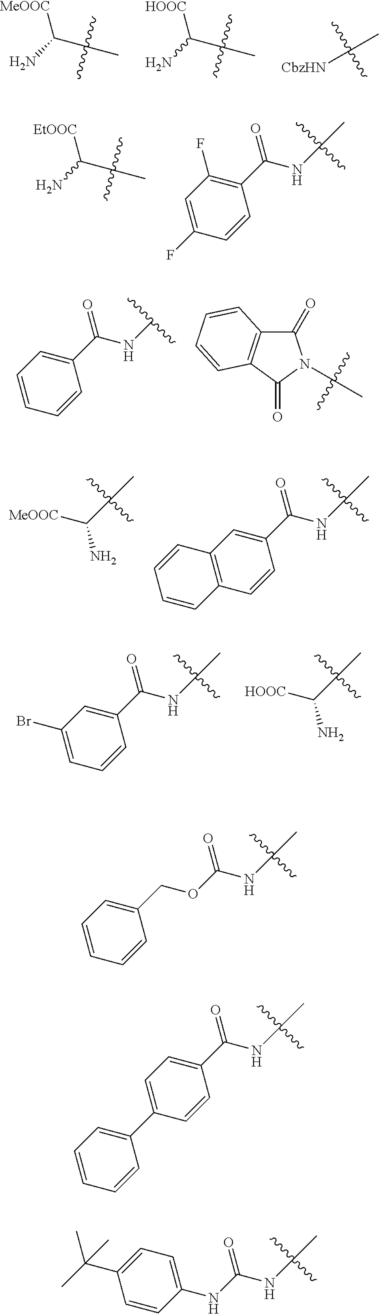
and wherein said compound is selective for DOT1L Methyl Transferase.

3. A compound of Formula III, a pharmaceutically acceptable salt thereof, a prodrug thereof, or combinations thereof: 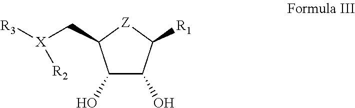
wherein R₁ is H, or a substituted or nonsubstituted: alkyl, cycloalkyl, morpholino, aryl, biaryl, fused biaryl, benzyl; heterocycle, purine, pyrimidine, alcohol, amine, amide, aldehyde, ketone, thiol; ester, ethers, carboxylate, acyl halide, imide, amidine, nitrile, cyano, thioaldehyde, ketone, thione, thioester, thioether, hydrazines, or disulphide;
Z is CH₂;
X is C, N, O or S; wherein if X=O, R₂=O;
R₃ is H, O, or R₁;
R₂ is H, O, or R₁;
or R₃ and R₂ are cyclized together to form a substituted or nonsubstituted: alkyl, cycloalkyl, aryl, biaryl, fused biaryl, benzyl; heterocycle, purine, pyrimidine; and wherein said substituent is selected from R₁, R₂, R₃, X, halide; or combinations thereof; and
wherein said compound is selective for DOT1L Methyl Transferase.

4. A compound of Formula IV, a pharmaceutically acceptable salt thereof, a prodrug thereof, or combinations thereof: 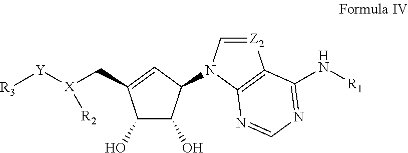
wherein R₁ is H, alkyl, or benzyl;
R₂ is H, 2-cyanoethyl, 2-methoxycarbonylethyl, methyl, 2-iodoethyl, ethanol, butyl, or benzyl carbamate;
X is N, C, or S; wherein if X=S, R₂=O; and wherein if X=C, R₂ is also equal to R₃ or R₁, and Y is also equal to R₁, R₂ or R₃;
Y is C₁, C₂, C₃ or C₄;
Z₂ is N, or CR₄, wherein R₄ can be a halogen, alkyl, aryl or a 5- or 6-membered heterocycle;
R₃ is H or selected from the following: 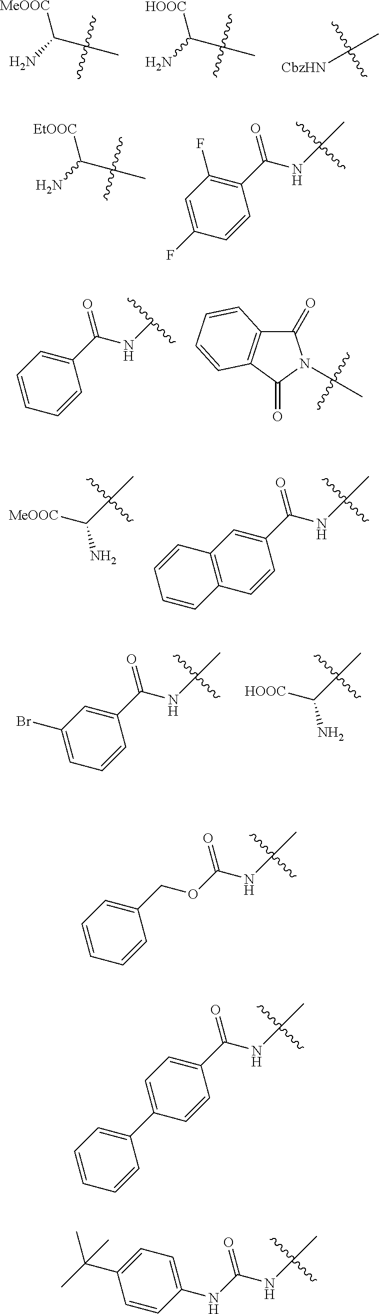
and wherein said compound is selective for DOT1L Methyl Transferase.

5. The compound of claim 1, wherein R₁ specifically binds in the hydrophobic pocket comprising Phe223, Leu224, Val249, Lys187 and Pro133 of DOT1L protein, thereby selectively inhibiting DOT1L Methyl Transferase activity.

6. The compound of claim 1, wherein the N6 hydrogen forms a hydrogen bond with Asp222 of the DOT1L protein; thereby selectively inhibiting DOT1L Methyl Transferase activity.

7. The compound of claim 1, wherein said compound has specificity for DOT1L and is substantially free of specificity for CARM1, PRMT1, G9a and SUV39H1 Methyl Transferases.

8. The compound of claim 1, wherein said compound specifically inhibits methylation of histone H3-lysine79 residues located in nucleosome core structure.

9. A composition comprising the compound of claim 1, and a pharmaceutically acceptable carrier.

10. A method of treating mixed lineage leukemia and/or breast cancer in a subject, comprising administering to the subject a therapeutically effective amount of the compound of claim 1.

11. The method of claim 10, wherein said compound is administered as a prodrug; wherein said prodrug comprises replacing RCOOH or RCONH₂ with an analogous alkyl ester, an aryl ester, or a heteroaryl ester.

12. A method of detecting mixed lineage leukemia and/or breast cancer comprising: adding a diagnostically effective amount of the compound of claim 1, to an in vitro biological sample.

13. A method of detecting mixed lineage leukemia and/or breast cancer in a subject, comprising administering to the subject a therapeutically effective amount of a compound of claim 1.

14. The method of claim 13, wherein said subject is human.

15. A method of targeting leukemia cells and/or breast cancer cells, comprising treating said cells with a compound of claim 1, wherein said cells comprise an elevated amount of DOTL1, compared to non-cancerous cells; and wherein said cells are in vitro or in vivo.

16. The method of claim 15, wherein the breast cancer cells comprise breast cancer stem cells.

17. The method of claim 15, wherein said targeting further comprises at least one of: inhibiting cell self-renewable ability and induces differentiation of breast cancer stem cells; and reversing disregulated gene expression of claudins, E-cadherin, and epithelial mesenchymal transition traits.

18. A method of treating mixed lineage leukemia and/or breast cancer, wherein a compound of Formula VI, a pharmaceutically acceptable salt thereof, a prodrug thereof, or combinations thereof: 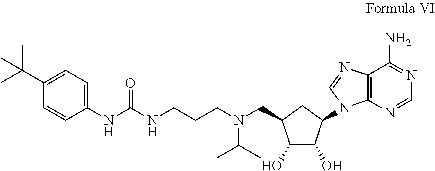 [SMILES: CC(C)N(CCCNC(=O)Nc1ccc(C(C)(C)C)cc1)C[C@H]1C[C@@H](n2cnc3c(N)ncnc32)[C@H](O)[C@@H]1O]
is administered to a subject, wherein said subject comprises cancer cells expressing a high level of DOT1L and wherein said compound selectively targets DOT1L.

19. A method of treating mixed lineage leukemia, wherein a compound of Formula VII, a pharmaceutically acceptable salt thereof, a prodrug thereof, or combinations thereof: 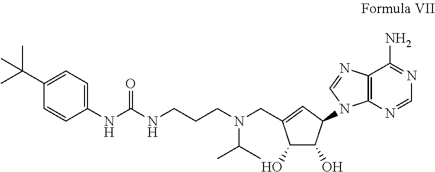 [SMILES: CC(C)N(CCCNC(=O)Nc1ccc(C(C)(C)C)cc1)CC1=C[C@@H](n2cnc3c(N)ncnc32)[C@H](O)[C@@H]1O]
is administered to a subject, wherein said subject comprises mixed lineage leukemia and wherein said compound selectively targets DOT1L.

20. Use of the compound of claim 1, in the manufacture of a medicament for the treatment of mixed lineage leukemia and/or breast cancer.

21. A method for the preparation of compounds of Formula II, pharmaceutically acceptable salts thereof, prodrugs thereof, or combinations thereof: 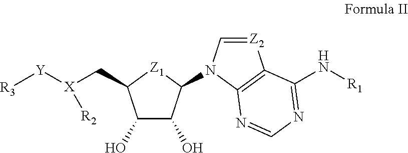
wherein R₁ is H, alkyl, or benzyl;
R₂ is H, 2-cyanoethyl, 2-methoxycarbonylethyl, methyl, 2-iodoethyl, ethanol, butyl, or benzyl carbamate;
X is N, C, or S; wherein if X=S, R₂=O; and wherein if X=C, R₂ is also equal to R₃ or R₁, and Y is also equal to R₁, R₂ or R₃;
Y is C₁, C₂, C₃ or C₄;
Z₁ is O, S, N, or CH₂;
Z₂ is N, or CR₄, wherein R₄ is a halogen, alkyl, aryl or a 5- or 6-membered heterocycle;
R₃ is H or selected from the following: 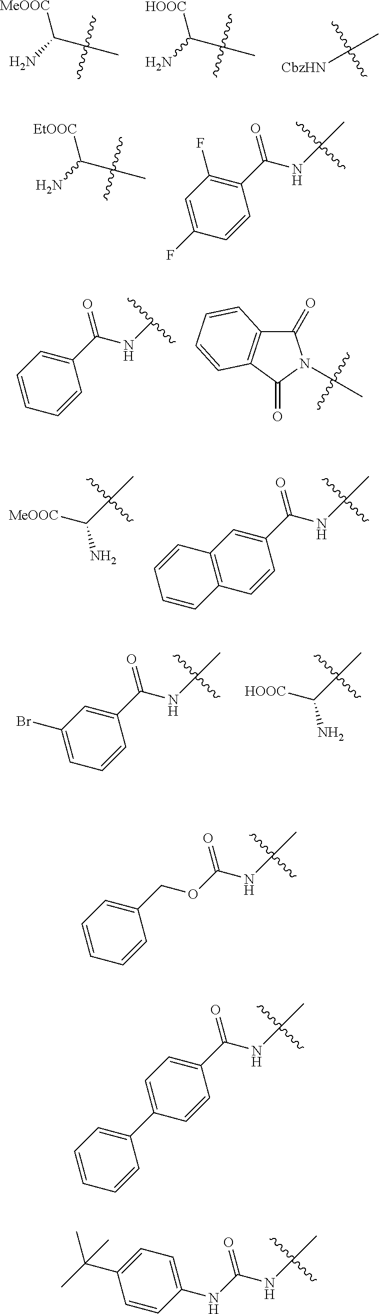
comprising reacting: 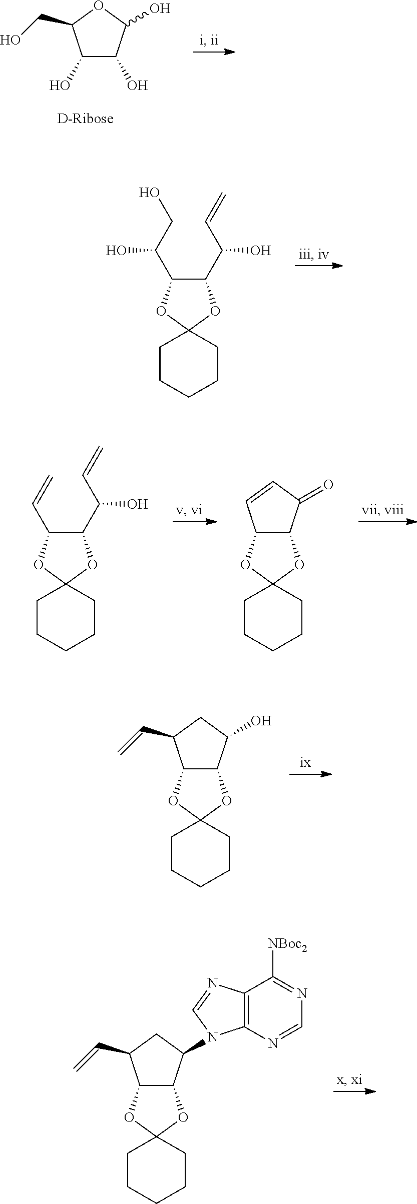 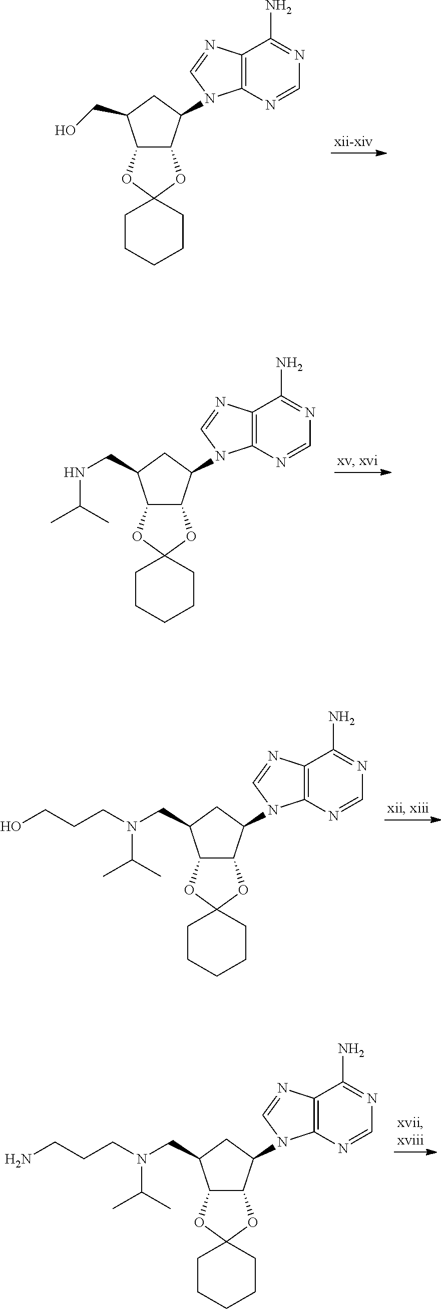 [SMILES: Nc1ncnc2c1ncn2[C@@H]1C[C@H](CO)[C@H]2OC3(CCCCC3)O[C@H]21 ; CC(C)NC[C@H]1C[C@@H](n2cnc3c(N)ncnc32)[C@@H]2OC3(CCCCC3)O[C@H]12 ; CC(C)N(CCCO)C[C@H]1C[C@@H](n2cnc3c(N)ncnc32)[C@@H]2OC3(CCCCC3)O[C@H]12 ; CC(C)N(CCCN)C[C@H]1C[C@@H](n2cnc3c(N)ncnc32)[C@@H]2OC3(CCCCC3)O[C@H]12] 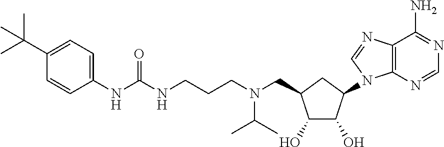 [SMILES: CC(C)N(CCCNC(=O)Nc1ccc(C(C)(C)C)cc1)C[C@H]1C[C@@H](n2cnc3c(N)ncnc32)[C@H](O)[C@@H]1O]
wherein: (i) cyclohexanone, cat. H₂SO₄; (ii) CH₂=CHMgBr, tetrahydrofuran (THF), −78° C.; (iii) NalO₄, MeOH/H₂O; (iv) Ph₃PCH₃Br, t-BuOK, THF; (v) 2^{nd} generation Grubbs' catalyst (5 mmol %), CH₂Cl₂; (vi) Dess-Martin periodinane (DMP), CH₂Cl₂; (vii) CH₂=CHMgBr, trimethylsilyl chloride (TMSCl), hexamethylphosphor-amide, CuBr.Me₂S, THF, −78° C.; (viii) NaBH₄, CeCl₃.7H₂O, MeOH, 0° C.; (ix) 6,6-di-Boc-adenine, Ph₃P, diisopropyl azodicarboxylate (DIAD), THF; (x) O₃, CH₂Cl₂, −78° C., then NaBH₄/MeOH; (xi) trifluoroacetic acid, CH₂Cl₂; (xii) phthalimide, PPh₃, DIAD, THF; (xiii) NH₂NH₂, EtOH, 80° C.; (xiv) acetone, NaCNBH₃, MeOH; (xv) Methyl acrylate, MeOH, 65° C.; (xvi) LiAlH₄, THF, −15° C.; (xvii) 4-^{t}BuPhNCO, CH₂Cl₂; and (xviii) HCl, MeOH.

22. A method for the preparation of compounds of Formula IV, pharmaceutically acceptable salts thereof, prodrugs thereof, or combinations thereof: 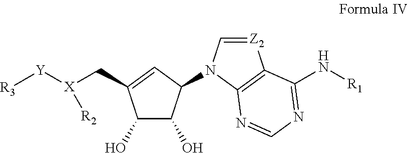
wherein R₁ is H, alkyl, or benzyl;
R₂ is H, 2-cyanoethyl, 2-methoxycarbonylethyl, methyl, 2-iodoethyl, ethanol, butyl, or benzyl carbamate;
X is N, C, or S; wherein if X=S, R₂=O; and wherein if X=C, R₂ is also equal to R₃ or R₁, and Y is also equal to R₁, R₂ or R₃;
Y is C₁, C₂, C₃ or C₄;
Z₂ is N, or CR₄, wherein R₄ can be a halogen, alkyl, aryl or a 5- or 6-membered heterocycle;
R₃ is H or selected from the following: 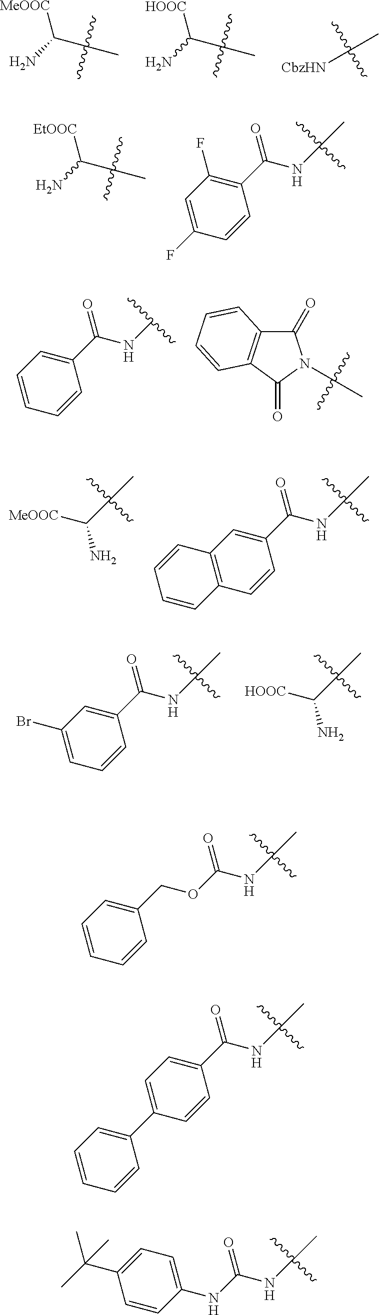
comprising reacting: 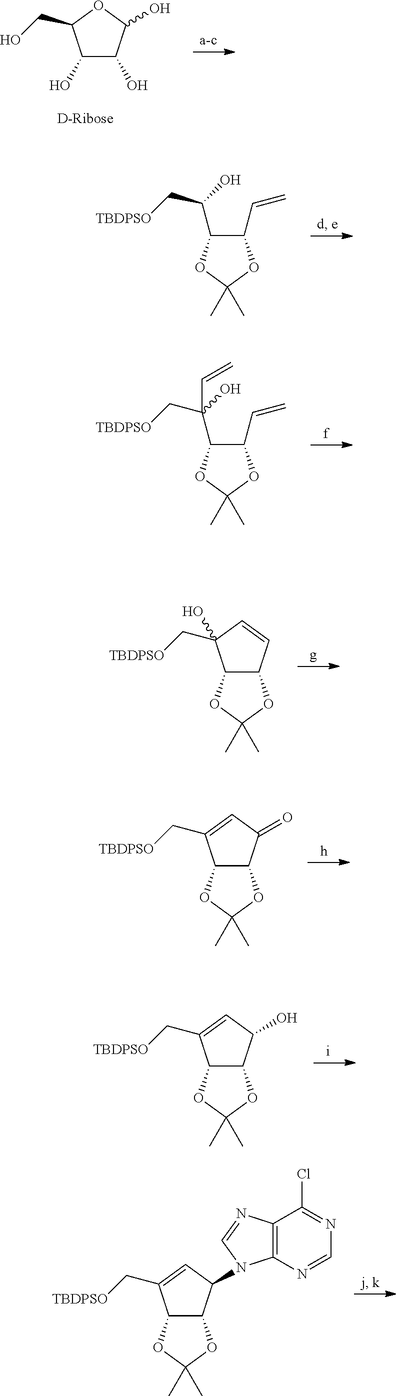 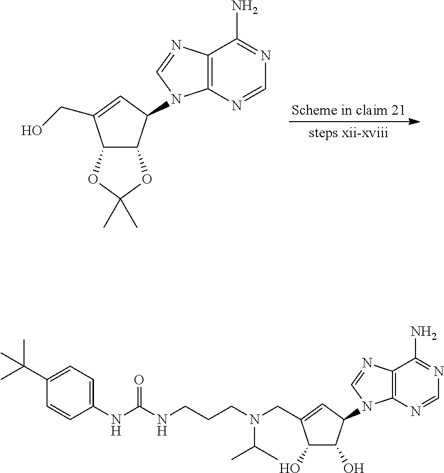 [SMILES: CC1(C)O[C@@H]2[C@H](O1)C(CO)=C[C@H]2n1cnc2c(N)ncnc21 ; CC(C)N(CCCNC(=O)Nc1ccc(C(C)(C)C)cc1)CC1=C[C@@H](n2cnc3c(N)ncnc32)[C@H](O)[C@@H]1O]
wherein: (a) acetone, cat. H₂SO₄; (b) tert-butyldiphenylsilyl chloride (TBDPSCl), Et₃N, 4-dimethylaminopyridine, dimethylformamide (DMF); (c) Ph₃PMeBr, t-BuOK, THF; (d) SO₃Py, Et₃N, CH₂Cl₂; (e) CH₂=CHMgBr, THF, −78° C.; (f) 2^{nd} generation Grubbs catalyst (5 mmol %), CH₂Cl₂, reflux; (g) pyridinium dichromate (PDC), 4 Å molecular sieve, DMF; (h) NaBH₄, CeCl₃.7H₂O, MeOH, 0° C.; (i) 6-chloropurine, Ph₃P, DIAD, THF; (j) 7 M NH₃ in MeOH, 100° C.; (k) tetrabutylammonium fluoride, THF; and (1) H₂, 10% Pd/C, MeOH.
